(19) United States
(12) Patent Application Publication
(10) Pub. No.: US 2023/0372032 A1
(43) Pub. Date: Nov. 23, 2023

(54) ROBOTIC ARTIFICIAL INTELLIGENCE NASAL/ORAL/RECTAL ENTERIC TUBE

(71) Applicant: The Brigham and Women's Hospital, Inc., Boston, MA (US)

(72) Inventors: William B. GORMLEY, Boston, MA (US); Brittany STOPA, Boston, MA (US)

(21) Appl. No.: 18/027,261

(22) PCT Filed: Sep. 21, 2021
(86) PCT No.: PCT/US2021/051376

§ 371 Date: Mar. 20, 2023

Related U.S. Application Data
(63) Continuation-in-part of application No. 17/027,364, filed on Sep. 21, 2020.

(51) Int. Cl. A61B 34/30; A61B 1/00; A61B 1/015; A61B 1/07; A61B 1/267; A61M 16/04; A61M 16/10

(52) U.S. Cl. CPC A61B 34/30; A61B 1/00165; A61B 1/015; A61B 1/07; A61B 1/00154; A61B 1/267; A61M 16/04; A61M 16/1005; A61B 2034/301

(57) ABSTRACT

A system and method by which a catheter tube may be automatically driven to a target location within the body of a subject, such as an enteral cavity or respiratory tract of the subject. The catheter tube may include an imaging device, a transceiver, a spectrometer, and a battery embedded in a tube wall at a distal end of the catheter tube. The imaging device may capture image data of structures proximal to the distal end of the catheter tube. An articulated stylet may be inserted in the catheter tube, which may be controlled by a robotic control engine according to navigation data generated by an artificial intelligence (AI) model based on the topographical image data. The spectrometer may sample and identify biomarkers proximal to the catheter tube. A remote computer may implement the robotic control engine and AI model and may wirelessly receive the image data from the transceiver.

### CROSS-REFERENCE TO RELATED APPLICATIONS

This application is based on, claims the benefit of, and claims priority to U.S. patent application Ser. No. 17/027,364, filed Sep. 21, 2020, which is hereby incorporated herein by reference in its entirety for all purposes.

### STATEMENT OF FEDERALLY SPONSORED RESEARCH OR DEVELOPMENT

The invention was made without any government support, partnership or grant.

### BACKGROUND OF THE INVENTION

This invention relates to methods and systems for placing a tube from an external surface of the body (e.g., nasal/oral/rectal) cavity into the enteral space anywhere from the stomach to the small or large intestine or the other body lumens and orifices, including the respiratory tract.

During the course of medical care, the need to access the enteral system is extremely common. For example, access to the enteral system may be needed to remove gastric or intestinal compounds, to introduce material into the enteral system or to obtain images or samples. Examples of removing gastric or intestinal compounds include gastric or intestinal decompression in the setting of gastric or intestinal paralysis, or in the setting of ingestion of toxic compounds. Examples of the need for introduction of material, which are more common, include feeding or providing medications to patients incapable of completing these activities independently. The need for imaging is common in both the upper and lower intestinal tract to observe and obtain samples from these areas. This includes the use of diagnostic esophago-gastro-duodenoscopy, and colonoscopy. This is generally accomplished through the manual placement of a nasogastric tube (NGT) or an orogastric tube (OGT), a rectal tube, or an endoscope for either the upper or lower intestinal tract. Accessing the enteric system can be accomplished rostrally or caudally.

A rostral approach to accessing the enteric system involves naso/oral access. The rostral approach will now be described.

The manual placement of naso/oral enteric tubes is a common procedure in the hospital setting and is crucial in treating patients with compromised oral intake. These manual placements are performed in multiple hospital settings, including the emergency room, the inpatient setting, and occasionally even in the outpatient setting. The use of these tubes is particularly common in the Intensive Care Unit (ICU) setting. It is estimated that over 1.2 million of these devices are placed annually in the United States alone. Although this procedure is performed frequently and considered generally to be simple, it does require a clinician with subject matter expertise to assist in the accurate manual placement of the device. Depending on institutional policy, the procedure may only be performed by a physician or a nurse with specialized skills.

The main concerns with the current model of naso/oral enteric tube placement are two-fold: (1) the safety of this placement for patients, and (2) the efficiency of the placement process.

Despite the presumed simplicity of the procedure for placing naso/oral enteric tubes, it is known to be a source of frequent and sometimes fatal complications. The worst of these complications come from the inadvertent placement of the tube into the respiratory tract with the potential complication including the introduction of feeding material into the lung, pneumonias, lung rupture with consequent pneumothorax and bronchopleural fistulas. All these complications can be fatal. The reason for these complications is that over 70% of naso/oral enteric tubes are manually placed blindly through the nose or mouth, traveling through the esophagus into the stomach or intestines. This blind placement is performed without any visual guidance, and many times results in erroneous tube placement and the resultant complications. In a minority of cases, these tubes are placed by highly specialized physicians, who are trained and equipped to use endoscopic or radiologic techniques to assist in the correct placement. However, involvement of such specialists is resource-intensive and creates a significant delay in accessing the enteral system of the patient. As a result, it is not ideal for either the healthcare system or the patient to utilize such resources for the placement of these devices.

In addition, there is often considerable discomfort for the patient associated with the placement of a naso/oral enteric tube. The inflexibility of the tube in conventional systems leads to difficulty navigating the complex pathway from the nose/mouth to the enteric system. As a result, the tube will often contact the patient's throat with some force, which can result in discomfort or injury.

Beyond the safety concerns of this procedure, the process of placing naso/oral enteric tubes tends to be inefficient. In a typical setting, the tube is manually placed blindly at the bedside by a clinician. Because of the potentially fatal complication of inserting the tube into the lung, the position of the tube in either the stomach or the intestine must be confirmed by radiology. For example, after the placement of a naso/oral enteric tube, a radiographic scan of the chest and abdomen must be obtained so that a trained radiologist or physician can confirm that the naso/oral enteric tube has been placed correctly. The need for this confirmation introduces a significant delay as radiographic scans are not always readily available. Once the radiographic scan has been performed, the scan must be reviewed by a radiologist or physician to verify correct distal tube placement before the tube can be utilized clinically. If the radiographic scan reveals that the tube is in the incorrect position, or if the position is not completely discernible, then the process must be repeated with a re-positioning of the tube and a repeat a radiographic scan. With this multi-step process, the patient's clinical needs for either enteral decompression, medication delivery, or initiation of feedings can be significantly delayed, on occasions up to 24 hours after the initiation of the process.

The use of such confirmatory resources, including radiology technicians and clinicians, bedside clinicians, radiographic imaging, and potentially additional naso/oral enteric tubes, can add considerable cost to this procedure, beyond the treatment delays it incurs.

The use of radiographic imaging introduces additional ionizing radiation to the patient, which is associated with known risks. Exposure to radiation causes damage to cells, tissues, and organs, which can lead to cancer and other medical problems. Efforts are underway across the healthcare industry to reduce exposure to radiation wherever possible, in order to protect patients from unnecessary risk. Specific populations are at increased risk of harm due to radiation, such as pediatric patients, patients who have undergone radiation therapy, patients who have been exposed to radiation from a nuclear accident, and people who live at high altitude.

While naso/oral enteric tubes with visual guidance exist that allow specialized clinicians to see the trajectory of the tubes, these devices require the specialized knowledge of the clinician, who in placing the device must be capable of discerning from captured images when the ideal position of the tube is reached. In institutions that have clinicians with this expertise, there will be a delay imposed by the availability of such experts. In institutions with no such expertise, this solution becomes non-operative. Furthermore, even with visual guidance, human mistakes in interpretations of the visual images and failed placement may still occur. It is widely understood that these tubes can be misplaced in error even when visual guidance is provided.

Furthermore, given the inevitability of tube migration within the enteric system and the need for maintaining proper positioning of the naso/oral enteric tube over a period, continued monitoring by an expert clinician is required. Conventional methods of naso/oral enteric tube placement rely on repeat radiographic scans to confirm tube placement periodically. Exposure to damaging radiation associated with radiographic imaging is increased with each additional scan obtained. The risk of damage due to feeding or delivering medications to the incorrect enteric location are increased when there is no indwelling localization mechanism associated with the naso/oral enteric tube.

Thus, an efficient and safe form of naso/oral enteric tube placement is needed.

Complex placement often requires the involvement of specialized endoscopes and endoscopically trained physicians for placement. This delays placement and given the enhanced complexity of the system, implies greater risk to the patients. Therefore, a safer, less complex, and automated way of guiding and imaging the upper intestinal tract is required. This will also allow for the automated system to obtain images and samples of the enteral system.

A caudal approach to accessing the enteric system involves rectal access. The caudal approach will now be described.

Similar to the issues with accessing the upper intestinal tract, placement of an enteral tube through the rectum is a blind and manual procedure. This has the potential complication of causing damage to the lower intestinal tract and misplacement. A safe, simple, automated system for placement of an enteral tube through the rectum into the lower intestinal tract is required that would allow for infusion of material, decompression of the large bowel, and acquisition of images and samples through an automated system not requiring specialized personnel to perform.

The main concerns with the conventional methods of rectal enteric tube placement are two-fold: (1) the safety of this placement for patients, and (2) the efficiency of the placement process.

As mentioned, the placement of rectal tubes for the purpose of large bowel decompression is performed manually and with no guidance. In other words this is performed as a blind procedure with the risk inherent in the unguided introduction of any device including creation of bleeding, intestinal rupture or misplacement This becomes even more severe if the placement of this tube is then followed by introduction of material such as those intended to facilitate intestinal decompression.

If the goal of tube placement is for obtaining images and tissue samples, the problem is different and more significant. In this case, the need for direct vision by a specialized physician such as a gastroenterologist necessitates the existence of highly specialized personnel and equipment. In this case, there exists the need for gastroenterologists capable of using an endoscopic device designed for use in the rectum and entire large bowel. The complexity of both the expert personnel and the equipment required to perform the procedure creates two major problems for the delivery of adequate patient care.

The first problem is patient access. Given the nature of expert personnel and equipment availability through the world, the access to needed diagnostic imaging capable of discerning lesions from within the enteral system is extremely limited. The recommendation for diagnostic colonoscopy for all patients above a certain age is severely constrained by the availability of these resources.

The second problem is that it is a complex procedure with significant patient discomfort and risk. The need for a physician to be able to visualize the entire colon in real time requires the use of large endoscopes developed for the exploration of the colon. This requires a full hospital procedure done under sedation/anesthesia in order to avoid patient discomfort. This is a combination of the fact that the equipment required is by necessity large and that the procedure can be prolonged. This combination creates the need for a full operative and anesthetic intervention with its significant increased costs and, importantly, with increased patient risk both from the procedure and the anesthetic.

These are not only dangerous, uncomfortable, costly and inefficient processes, they also limit needed care. A system that improves upon these common problems would provide value and benefit to patients, clinicians, and healthcare systems.

### SUMMARY OF THE INVENTION

Another type of tube placement that poses difficulty in the medical setting involves access to the respiratory tract. The need to enter the respiratory tract, specifically, the lungs, is often in an emergent situation where need to control the patient's breathing is paramount. In these situations, the failure to gain access to the lungs traversing though the body's natural pathways represented by the oral/nasal space, pharynx, glottis, and into the trachea can be fatal. Establishing access to the trachea and lungs allows for a patient to be ventilated and oxygenated.

The respiratory tract approach will now be described.

Similar to the issues with accessing the enteric system, placement of an endotracheal intubation tube for respiratory tract access is a complex manual procedure. This has the potential complication of causing damage to the lips, teeth, oral cavity, epiglottis, larynx, or trachea, in addition to the issue of misplacement or failed placement. In either of these latter two scenarios of misplaced or failed placements, the urgent need to ventilate and oxygenate a patient remain unresolved. A safe, simple automated system for placement of an endotracheal intubation tube through the oropharynx into the trachea is required which would allow for ventilation, oxygenation, airway management, and respiratory support. Currently this is done through a manual procedure, performed by highly skilled and trained individual. Proposed herein are solutions which utilize the automated system described in this document for accessing the enteric system and which would not require medical personnel to perform.

The need for such an automated intubation system is particularly valuable when a patient is encountered outside of standard hospital settings, specifically, in the field where first responders and medics often encounter traumatically injured patients. In the setting of trauma, the most important concern is always patient safety. Often patients have lost all control of basic physiologic functions, most important of which are maintenance of a functional airway, the ability to breathe and the need to maintain adequate circulation. These three are immortalized in the ABC's of care—Airway, Breathing and Circulation. Traumatic injuries are often accompanied by a loss of a viable airway and with it, the ability for a patient to continue providing oxygen to at risk tissues - making establishing a viable airway critical.

The development of a compact, portable, fully autonomous robotic device capable of providing an emergency airway to all our trauma patients will have a significant impact on the level of recovery and survival of these patients. This can be accomplished using visual based data and advanced data analytics and artificial intelligence to drive the device that allows for early, safe and dependable endotracheal intubation. Outcomes in trauma are determined by decision and actions made in the first 30-60 minutes of care and delivering advanced assistance to care givers as rapidly as possible at the point of care will improve outcomes for patients involved in such situations.

Currently only a minority of both civilian or military first medical responders are capable of advanced airway control by securing an airway with endotracheal intubation. This is a significant problem especially in patients suffering cardio-respiratory arrest, traumatic brain injury or facial, neck or chest wounds. Studies have demonstrated high levels of complications when advanced airways are attempted in non-hospital settings, such that this is not a skill that is required for Emergency Medical Technician certification. It is however critical to provide an adequate airway to all of these patients in the first hour of their injury or cardio-pulmonary failure, what is known as the “golden hour” because of the critical role determined by decision and actions made in the first 30-60 minutes of care. Delivering advanced assistance to care givers as rapidly as possible at the point of care will improve outcomes for patients involved in such situations.

The main concerns with the conventional methods of endotracheal intubation tube placement are three-fold: (1) the safety of this placement for patients, (2) access to care based on the complexity and the efficiency of the placement process, and (3) risk to medical personnel:
1) Patient Safety: The placement of endotracheal tubes for the purpose of airway management and respiratory support is performed manually and with visual guidance through the use of various devices including a direct laryngoscope or video laryngoscopy. On occasion these tubes can be placed blindly using nasal endotracheal intubations. In any of these approaches, visualization is limited to varying extents leading to the risks inherent in the unguided introduction of any device including bleeding, tissue rupture, and, importantly, misplacement or even failed placement. Given these difficulties it is not uncommon to see prolonged intubation attempts during which the patient is subjected to life-threatening periods of lack of pulmonary ventilation and tissue oxygenation.
2) Access to Care. The complexity of the procedure and the associated risks necessitate that a highly trained and specialized clinical team be present to perform the intervention. The need for a sophisticated team of medical personnel for tracheal access and securing of an airway is particularly problematic, especially since the setting in which this is required are often emergent and life threatening. Inability to resolve respiratory failure in this setting by effectively intubating a patient can be fatal. This is a special concern with patient in a non-clinical setting such as a patient's home, the battle field, or an emergency transport vehicle. This lack of patient access to this life saving procedure is one of the major problems with the current system of endotracheal intubation.
3. Risk to medical personnel. During endotracheal intubation tube placement, given the nature of the placement access point in the oral/nasal and pharyngeal cavities, the patient is likely to release airborne droplets. This process, known as aerosolization, is considered the point during which all healthcare person now in the immediate vicinity of the procedure are the highest lists risk of contagion with any pathogen that the patient may have. Given the fact that respiratory failure often is associated with infectious causes such as viral or bacterial pneumonias, this is a critical safety risk for healthcare personnel involved with the procedure of intubation. Limiting exposure to infection transmission is critical in all settings, but particularly so in the setting of a respiratory pandemic such as COVID-19 infectious transmission. To protect the medical personnel involved in the procedure from this infectious transmission, personal protective equipment (PPE) is employed. However the considerable size of the medical team needed for this blinded, manual endotracheal intubation puts a number of medical personnel at risk of infection. This risk exists when treating all hospitalized patients, but is especially great during a global pandemic of a respiratory illness such as what the world is currently experiencing with COVID-19.

Manual endotracheal intubation is not only a dangerous, uncomfortable, costly, and inefficient process, it also puts medical personnel at risk of airborne infectious transmission.

Thus there is a need for an automated endotracheal intubation solution that would limit the exposure of medical personnel to such risk of infection.

Systems and methods for the automated placement of a catheter tube at a target location within the body of a subject are disclosed. For example, a catheter tube may be automatically navigated and driven into the enteral space, from either a rostral approach from the nasal/oral cavity, from a caudal approach from the rectum, or from the oral cavity to the respiratory tract. This automatic navigation may be performed using a robotic mechanical device guided by artificial intelligence models to create a “self-navigating” catheter. A system performing such placement may not require the intervention of a clinician and therefore may eliminate the need for specific expertise for the positioning or placement of the device into the enteral system or respiratory tract of a subject (e.g., patient).

The system may therefore enable directed placement and immediate confirmation of correct positioning of the tube using topographic imaging data captured by one or more image sensors of the system and corresponding to the cavity in which the tube is positioned. The embodiments described herein may be applied for naso- and oro-enteric tube placements, rectal enteric tube placements as well as the placement of percutaneous feeding tubes, and placement of endotracheal intubation tubes. It should be understood, however, that the described embodiments are intended to be illustrative and not limiting. For example, embodiments described herein are not limited in any way to a particular port of entry to access the enteral system or respiratory tract, or to the final position of the tube itself.

This system will also make possible the acquisition of imaging data and/or samples from within the cavity. It should be understood that imaging data described herein may refer to imaging data acquired through one or more (e.g., multimodal) sources and/or acquired using one or more imaging techniques, examples of which will be described below.

The system may employ artificial intelligence models for processing the data input from the imaging sensors, which may enable both “self-navigating” catheter placement as well as subsequent enteral or respiratory environment calculations.

The system may furthermore remain indwelling in the patient as clinically indicated. In this way, the data obtained from the sensors in the distal catheter may be utilized by the clinical team for continuous monitoring of the enteral or respiratory environment. This monitoring may include catheter localization information and, in the enteric system, biomarkers and pH metrics, and enteric volume measures.

The embodiments described herein may be applied for naso- and oro-enteric percutaneous feeding tubes as well as the placement of rectal tubes, respiratory tract access via endotracheal intubation, and their subsequent monitoring, imaging and sampling capabilities. It should be understood, however, that the described embodiments are intended to be illustrative and not limiting. For example, embodiments described herein are not limited in any way to a particular port of entry to access the enteral system or respiratory tract, or to the final position of the tube itself.

In an example embodiment, a system may include a catheter tube that includes a tube wall that defines a lumen, an imaging device configured to capture image data, the imaging device disposed at a distal end of the catheter tube, a transceiver coupled to the imaging device and configured to wirelessly transmit the captured image data, the transceiver disposed at the distal end of the catheter tube, an articulated stylet disposed in the lumen of the catheter tube, the articulated stylet comprising an articulated distal end, a robotic control and display center. The robotic control display center may include wireless communication circuitry that communicates with and receives the image data from the transceiver, processing circuitry configured to execute an artificial intelligence algorithm that analyzes the image data and outputs corresponding navigation data, and a robotic control engine that drives the articulated stylet toward a target destination inside a body of a subject based on the navigation data.

In some embodiments, the imaging device may be a topographic imaging device, and the captured image data may include topographic image data.

In some embodiments, the imaging device may be a visual imaging device, and the captured image data may include still imaging data or visual image video data.

In some embodiments, the imaging device and the transceiver may be embedded in the articulating stylet. The articulated stylet may also include an insufflating channel embedded in the articulated stylet and a light source embedded in the articulated stylet.

In some embodiments, the imaging device and the transceiver may be embedded in the tube wall of the catheter tube. The catheter tube may further include an insufflating channel embedded in the tube wall of the catheter tube. The catheter tube may further include a light source embedded in the tube wall of the catheter tube.

In some embodiments, the imaging device may include a time-of-flight imaging device, the captured imaging data may include time-of-flight imaging data, and the time-of-flight imaging device may be configured to capture the time-of-flight image data using multiple wavelengths of light.

In some embodiments, the processing circuitry may be configured to execute a volume sensing module configured to obtain volume measurements of an enteral space, respiratory tract, or other cavity in which the catheter tube is disposed based on time of flight imaging using multiple wavelengths of light. The volume sensing module may, based on the volume measurements, determine a first volume value corresponding to a total volume of the enteral space, a second volume value corresponding to a first portion of the total volume that is empty, and a third volume value corresponding to a second portion of the total volume that is filled with material. The third volume may be calculated by subtracting the second volume from the first volume.

In some embodiments, the robotic control engine may be configured to drive the articulated stylet by controlling at least one articulation of the articulated stylet to control a direction of movement of the articulated stylet, the articulated stylet having at a minimum three degrees of freedom including plunge, rotation, and tip deflection.

In some embodiments, the catheter tube may further include a stylet spectrometer and a stylet transceiver disposed at the distal end of the articulating stylet. The stylet spectrometer may be configured to sample and analyze substances at the distal end of the articulating stylet to produce stylet spectrometer data and the stylet transceiver may be configured to wirelessly transmit the stylet spectrometer data to the robotic control and display center.

In some embodiments, the catheter tube may further include a spectrometer disposed in the distal end of the catheter tube, the spectrometer being configured to collect and analyze samples to produce spectrometer data.

In some embodiments, the robotic control and display center may include a display device. The transceiver may be configured to send the spectrometer data to the processing circuitry via the wireless communication circuitry. The processing circuitry may be configured to analyze the spectrometer data to identify a biomarker to which the sample corresponds. The display device may be configured to display information related to a location and a status of the catheter tube and information related to the biomarker.

In some embodiments, the at least one artificial intelligence model may include a detection and tracking model that processes the captured image data in near-real time, a deep-learning detector configured to identify orifices and structures within the enteral cavity or respiratory tract, the deep-learning detector including at least one convolutional-neural-network-based detection algorithm that is trained to learn unified hierarchical representations, that identifies the orifices and structures based on the captured image data, and that calculates the navigation data based on the captured image data and the target destination, and a median-flow filtering based visual tracking module configured to predict the motion vector of the articulated stylet using sparse optical flow.

In an example embodiment, a robotic control and display center may include wireless communication circuitry that communicates with and receives topographical image data from a transceiver of a catheter tube, processing circuitry configured to execute an artificial intelligence model that analyzes the topographical image data and a target destination and outputs corresponding navigation data, and a robotic control engine that automatically drives an articulated stylet disposed inside the catheter tube toward the target destination inside a body of a subject based on the navigation data.

In some embodiments, the robotic control engine may be configured to control a direction of movement of the articulated stylet by controlling an articulation in a distal end of the articulated stylet.

In some embodiments, the robotic control engine may be configured to control a direction of movement of the articulated stylet by modifying a rotational position of the articulated stylet.

In some embodiments, the wireless communication circuitry may be configured to receive spectrometer data from the transceiver, the spectrometer data corresponding to a substance sampled by a spectrometer of the catheter tube. The processing circuitry may be configured to execute an additional artificial intelligence model that receives the spectrometer data and outputs an identity of a biomarker to which the substance corresponds.

In some embodiments, the robotic control and display center may further include a display device that is configured to display information related to a location and status of the catheter tube and the identity of the biomarker.

In some embodiments, the robotic control engine may be configured to drive the articulated stylet without receiving manual guidance.

In an example embodiment, a catheter assembly may include a catheter tube and an articulated stylet. The catheter tube may include a tube wall that defines a lumen, an imaging device configured to capture image data, the imaging device disposed at a distal end of the catheter tube, and a transceiver coupled to the imaging device and configured to wirelessly transmit the captured image data to a remote computer system, the transceiver being disposed at the distal end of the catheter tube. The articulated stylet may be disposed in the lumen, and may be configured to be automatically driven to a target location within a subject based on at least the captured image data.

In some embodiments, the articulated stylet may include an articulation, the articulation being configured to bend to control a direction of motion of the articulated stylet while the articulated stylet is being automatically driven to the target destination.

In some embodiments, the articulation of the articulated stylet may possess at least three degrees of freedom comprising plunge, rotation, and tip deflection.

In some embodiments, the catheter tube may further include a spectrometer disposed at the distal end of the catheter tube, the spectrometer being configured to sample and analyze substances proximal to the distal end of the catheter tube to produce spectrometer data. The transceiver may be configured to wirelessly transmit the spectrometer data to the remote computer system.

In some embodiments, the imaging device, the spectrometer, and the transceiver may each be embedded at different locations in the tube wall of the catheter tube. The catheter tube may further include an insufflation channel embedded in the tube wall.

In some embodiments, the image data may include topographical image data depicting structures proximal to the imaging device.

Some embodiments of the disclosure provide a guidance system. The guidance system can include an illumination source configured to illuminate an interior of the patient, a robot system including an imaging device, and a stylet configured to be inserted into an orifice of a patient. The stylet can have a proximal end and a distal end. The stylet can include an optical bundle having an optical fiber optically coupled to the imaging device. The optical fiber can be configured to direct light from within the interior of the patient and to the imaging device.

In some embodiments, a stylet can include a plurality of filaments coupled to or integrated within a body of the stylet. A robot system can include a plurality of actuators. Each filament can be coupled to an extender of a respective actuator. Extension and retraction of the extender of the actuator tensilely loads the respective filament to adjust the orientation of the stylet relative to the robot system.

In some embodiments, a robot system can include a motor that can be configured to rotate the stylet to advance a distal end of the stylet further into the patient.

In some embodiments, a stylet can include a CO2 sensor. A robot system can include a controller in communication with the CO2 sensor. The controller can be configured to receive, using the CO2 sensor, a CO2 amount value, and determine that a distal end of the stylet is at a target location within the patient, based on the CO2 amount value.

In some embodiments, a controller can be in communication with the illumination source and the imaging device. The controller can be further configured to cause the illumination source to emit light to illuminate the interior of the patient, receive, using the imaging device, an image of the interior of the patient, identify an anatomical region of interest within the image, determine a desired orientation based on the identification of the anatomical region of interest within the image, cause the plurality of actuators to adjust the stylet to be oriented at the desired orientation, and advance the stylet further into the interior of the patient.

In some embodiments, a controller can be configured to receive, using the imaging device, another image of the interior of the patient, identify a tracheal bifurcation within the another image, and determine that the distal end of the stylet is at the target location within the patient, based on the CO2 amount value exceeding a threshold value, and the identification of the tracheal bifurcation within the another image.

In some embodiments, a stylet can include a channel. A robot system can include a gas source that can be configured to be in fluid communication with the channel. Gas from the gas source can be configured to be directed though and out the channel into the interior of the patient.

In some embodiments, a stylet can include a channel. A robot system can include a vacuum source that can be configured to be in fluid communication with the channel. The vacuum source can draw fluid out from the interior of the patient and through and out the channel.

In some embodiments, a stylet can include a light pipe optically coupled to the illumination source. The light pipe can direct light emitted from an illumination source into the interior of the patient. A lens can be optically coupled to a distal end of an optical fiber. The lens can be configured to focus light from within the patient into the distal end of the optical fiber.

In some embodiments, a stylet can include a channel, and a light pipe optically coupled to the illumination source. The illumination source can be part of a robot system. The stylet can include a CO2 sensor. An optical bundle, the light pipe, and the CO2 sensor each can be positioned within the channel.

In some embodiments, a guidance system can include an oropharyngeal device that can be configured to be inserted into the mouth of the patient.

In some embodiments, an oropharyngeal device can include a handle and a mouthpiece coupled to the handle. The handle can have a cross-sectional height that is greater than a cross-sectional height of the mouthpiece. The mouthpiece can have a curved section that curves away from a longitudinal axis of the oropharyngeal device. The mouthpiece can be configured to be positioned inside the mouth of the patient when the oropharyngeal device is placed into the orifice of the patient. The handle can be configured to be positioned outside of the mouth of the patient when the oropharyngeal device is placed into the orifice of the patient.

In some embodiments, a mouthpiece of an oropharyngeal device can be configured to contact a tongue of the patient. A distal end of a mouthpiece can be configured to be positioned within the throat of the patient.

In some embodiments, an oropharyngeal device can include a conduit extending through a handle and through a mouthpiece, and a port connector configured to interface with an oxygen gas source. The port connector can be in fluid communication with the conduit. Oxygen gas from the oxygen gas source can be configured to flow into the port connector, through and out the conduit into the throat of the patient.

In some embodiments, the guidance system can include an endotracheal tube. A distal end of the endotracheal tube can be configured to be inserted into the mouth and throat of the patient. The endotracheal tube can be configured to be removably coupled to the oropharyngeal device and a securing device that can be configured to be coupled to the head of the patient.

Some embodiments of the disclosure provide a method of intubating a patient. The method can include inserting a distal end of an oropharyngeal device into the mouth of the patient and into the throat of the patient, advancing a distal end of an endotracheal tube along the oropharyngeal device until the distal end is positioned within the throat of the patient, coupling the endotracheal tube to the oropharyngeal device, and inserting a distal end of a stylet into the endotracheal tube until the distal end of the stylet reaches a target location inside the trachea of the patient.

In some embodiments, a method can include decoupling an endotracheal tube from an oropharyngeal device, advancing a distal end of the endotracheal tube along a stylet until the distal end of the endotracheal tube overlaps with or is proximal to a distal end of the stylet, retracting the stylet back through the endotracheal tube until the entire stylet is outside of the patient, and engaging a ventilator with the proximal end of the endotracheal tube.

In some embodiments, a method can include introducing oxygen gas, from a pressurized oxygen gas source, through a port connector of an oropharyngeal device, through a conduit of the oropharyngeal device, and into the throat of the patient during an insertion of a stylet into an endotracheal tube until a distal end of the stylet reaches a target location.

### BRIEF DESCRIPTION OF THE DRAWINGS

FIGS. 1A through 1E illustrate an exemplary catheter tube that includes a power source, a spectrometer, a transceiver, and an imaging device, and a channel that may or may not be used for insufflation of the gastro-intestinal tract during placement of the device.

FIG. 2 illustrates an exemplary block diagram of circuitry within a distal end of a catheter tube, the circuitry including a power source, a spectrometer, a transceiver, and an imaging device, and a channel that may or may not be used for insufflation of the gastro-intestinal tract during placement of the device.

FIG. 3 illustrates a placement of an exemplary catheter tube within the stomach of a subj ect.

FIGS. 4A and 4B illustrate an exemplary robotic control and display center (RCDC) that may guide a catheter enteric tube to a target location within the enteral system or respiratory tract of a subject via an articulated stylet having an articulated end that is controlled according to navigation data generated by an artificial intelligence model to direct the tube to the target location in an enteral cavity or respiratory tract.

FIGS. 5A through 5C illustrate an exemplary catheter enteric tube into which an articulated stylet has been inserted. FIG. 5B illustrates a range of motion of the articulated stylet.

FIG. 6 illustrates a process flow for an exemplary method of automatically driving a catheter tube to a target destination using navigation data derived by one or more artificial intelligence models from image data generated by an imaging device disposed at a distal end of the catheter tube.

FIG. 7 illustrates an isometric view of an exemplary robotic control system that may manipulate a catheter tube into a desired location.

FIGS. 8A through 8B illustrates respective isometric and top views of a carriage that may be included in a robotic control system, which may contain an imaging device coupled to an articulated stylet, and which may rotate or articulate the articulated stylet.

FIG. 9 illustrates an exemplary cross section of an articulated stylet which may feature an image guide, light pipe, and a bending section with a pull wire for articulation.

FIG. 10 illustrates an exemplary articulated stylet that has been inserted into a catheter tube, and which includes proximal connection ports that may enable enteral access and/or insufflation, and which includes a proximal optical connector for imaging.

FIG. 11 illustrates a view of an exemplary articulated stylet that has been driven to a target location through the enteral space of a portion of a subject's intestines.

FIGS. 12A, 12B, and 12C illustrate the robot advancing the catheter device with its inner articulated stylet and the exterior catheter tube, demonstrating the self-driving robot advancing to the inflection point of the larynx where the robot can turn either anteriorly into the larynx and trachea or posteriorly into the esophagus.

FIG. 13 shows a schematic illustration of a block diagram of a guidance system.

FIG. 14 shows a schematic illustration of a side view of a stylet.

FIG. 15 shows an axial cross-sectional view of the stylet of FIG. 14 taken along line 15-15 of FIG. 14.

FIG. 16 shows a longitudinal cross-section of the stylet of FIG. 14 taken along line 16-16 of FIG. 14.

FIG. 17 shows a top isometric view of a robot system in an open configuration.

FIG. 18 shows a top isometric view of the robot system of FIG. 17 in a closed configuration.

FIG. 19 shows a top view of the robot system of FIG. 17 in the open configuration.

FIG. 20 shows a top view of the robot system of FIG. 17 in the closed configuration.

FIG. 21 shows a front isometric view of a securing device.

FIG. 22A shows a front isometric view of the oropharyngeal device.

FIG. 22B shows a rear isometric view of the oropharyngeal device of FIG. 22A.

FIG. 23 shows a flowchart of a process for guiding a stylet to a target location within a patient.

FIG. 24 shows a schematic illustration of an endotracheal tube inserted into the throat of the patient and with a stylet having been inserted into the endotracheal tube.

FIG. 25 shows a system configuration in which the image detection computer receives the visual feedback from the robot's camera located on the distal end of the stylet. The image detection algorithm processes the data and recognizes the anatomical landmarks of the image. This data is sent to the robotic control computer where the robotic control algorithm processes instructions for movement according to the anatomic location of the robot's stylet. The robot is controlled by the robot control computer via ROS communication. The robot control computer receives input from image detection computer to adjust control.

FIG. 26A provides an example of a robot guidance algorithm and FIG. 26B provides an example of a robot control algorithm.

FIG. 27 shows a diagram of a robot control scheme, in particular a schematic drawing of the robot control algorithm. The robot is controlled based on the tracking information received from the image detection computer.

### DETAILED DESCRIPTION

Systems and methods disclosed herein relate to automated placement of a catheter tube at a target location within the body of a subject (e.g., into the subject's enteral system via the subject's nose, mouth, or rectum, into the respiratory tract via the nasal or oral cavity, or via a surgical incision that extends to the subject's stomach or intestine directly). The catheter tube may further include a channel that may or may not be used for insufflation of the gastro-intestinal tract or respiratory tract during placement of the device. The catheter tube may include an imaging device, which can be a topographical imaging device that captures topographical images of structures in the vicinity of the distal end (e.g., tip) of the catheter tube, and/or a visual imaging device that captures pictures or videos from within the enteral cavity. Imaging data generated by such imaging devices may be topographical image data, still image data, video data, or a combination of some or all of these. The catheter tube may further include an image guide and light guides that can connect to a camera or spectrometer that is disposed outside the subject, which may be used to perform optical analysis of enteral spaces or respiratory tract of the subject. The catheter tube may further include a spectrometer, which may analyze biomarkers or other chemicals in the vicinity of the distal end of the catheter tube (e.g., such as biomarkers in tissue around the tip of the catheter tube). The catheter tube may further include a transceiver, which may wirelessly transmit and receive data to and from a remote device. The transceiver and wireless communication circuitry of the remote device may communicate using a wireless personal area network (WPAN) according to a short-wavelength UHF wireless technology standard, such as Bluetooth®, for example. It should be understood that other WPAN standards, such as ZigBee®, may instead be used in some embodiments. The remote device that communicates with the transceiver of the catheter tube may be a Robotic Control and Display Center (RCDC), which may include a display, an articulated stylet, a robotic control engine, processing circuitry, and wireless communication circuitry. The articulated stylet may be an articulated robotic navigating articulated stylet dimensioned to be placed within the catheter tube. The robotic control engine may drive the articulated stylet and may control its direction, so that the articulated stylet, and therefore the catheter tube, may automatically navigated through an opening in a subject's body (e.g., the nose or mouth of the subject) to a target location within the subject's body. One or more artificial intelligence (AI) models may be implemented by the processing circuitry of the RCDC. The AI model (s) may include one or more trained machine learning neural networks, which operate on image data received from the imaging device of the catheter tube via the transceiver to determine the direction in which the robotic control engine will drive the articulated stylet and catheter tube. The display may be a digital display screen, and may display information regarding the placement of the distal end of the catheter tube in the subject's body, continuously updated status information for the catheter tube, and biomarker information collected by the spectrometer of the catheter tube.

An artificial intelligence based detection and tracking model may be executed to enable the RCDC to traverse autonomously, and may use real-time captured enteral images (e.g., represented via topographic image data, still image data, and/or video data) or other sensor data, which may be captured by one or more imaging devices disposed at a distal end of an articulated stylet/catheter tube. The objective may be to first detect the nasal/oral/rectal opening from the enteral or respiratory tract images and then follow a path predicted by a detection-tracking based mechanism. For detection, a deep-learning YOLO-based detector may be used to detect the nasal/oral/rectal orifice, environmental features, and structures within the enteral cavity or respiratory tract. For example, the deep-learning YOLO-based detector may further distinguish between a nasal/oral/rectal orifice and visually similar nearby structures. For example, once inside the enteral cavity or respiratory tract, the deep-learning YOLO-based detector may subsequently discriminate between visually similar structures over the course of the path to the enteral or tracheal target. For tracking, a fast and computationally efficient median filtering technique may be used (e.g., at least in part to predict the motion vector for the articulated stylus in order to navigate the articulated stylus to a target destination).

For detection of orifices, structures, and surrounding environment, a convolutional neural network (CNN) based detector may be used in conjunction with the deep-learning YOLO-based detector (e.g., which may be collectively referred to as a “deep-learning detector”), as it has achieved a state-of-the-art performance for real-time detection tasks. Different from traditional methods of pre-defined feature extraction coupled with a classifier, these CNN-based detection algorithms may be designed by a unified hierarchical representation of the objects that are learned using imaging data. These hierarchical feature representations may be achieved by the chained convolutional layers which transform input vector into a high dimensional feature space. For enteral or tracheal detection, a 26-layer or greater CNN based detection model may be employed. In such a model, the first 24 layers may be fully convolutional layer that are pre-trained on Imagenet dataset, and the final two layers may be fully connected layers which output the detected regions. The algorithm may further be fine-tuned with colored images of the enteric regions.

For tracking, a median-flow filtering based visual tracking technique (e.g., performed by a median-flow filtering based visual tracking module) to predict the motion vector for the robotic placement device may be employed. The median flow algorithm may estimate the location of an object with sparse optical flow, and the tracking based system may be based on the assumption that an object consists of small and rigidly connected blocks or parts which more synchronously together with motion of the whole object. In some embodiments, the object may be the nasal orifice, oral orifice, rectal orifice, or structures within the enteric cavity or respiratory tract. Initialization of the algorithm may be performed by setting up a bounding box in which the enteral/tracheal cavity is located at first, and within this region of interest a sparse grid of points may be generated. The motion of the enteral/tracheal cavity detected by optical flow in the captured images may be computed as the median value of differences between coordinates of respective points that are located in the current and preceding images. Only those points which have been regarded as reliable during the filtering may be taken into account. The algorithm may be capable of estimating the object scale variations.

For implementation, the object detection may be accomplished via YOLO-based algorithm and object tracking may be accomplished via median flow tracker (e.g., which may be implemented through Python). The environment may be built on Ubuntu, for example. The graphics processing unit (GPU) integration cuDNN and CUDA toolkit may be used to implement these algorithms/models.

The training segment may be implemented by supplying annotated images to a Keras implementation of YOLO. The Keras and TensorFlow backend may be used. The dataset may be created with annotated software VoTT (Microsoft, Redmond, WA), with an adopted learning rate of 103 for 1,000 training epochs and saved model parameters every 100 epochs. Among the saved models, the one that achieves the highest Average Precision (AP) for Intersection over Union (IoU) of 50% or higher considered as positive on the validation set may be selected as the final model to be evaluated on the training set.

The detection segment may again be implemented based on Keras running TensorFlow on the backend. For tracking, the tracking API in OpenCV may be used. The bounding box may be detected by YOLO and passed to Median Flow tracker at m:n ratio, in order to realize real-time detection and tracking.

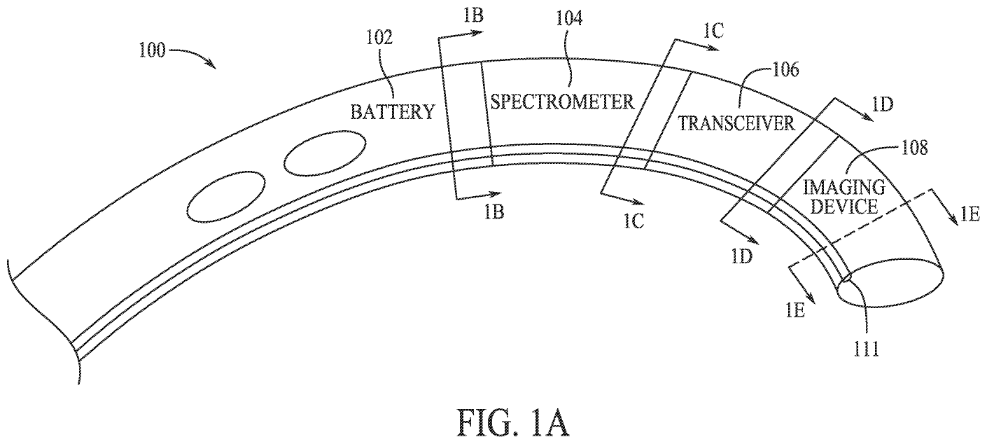

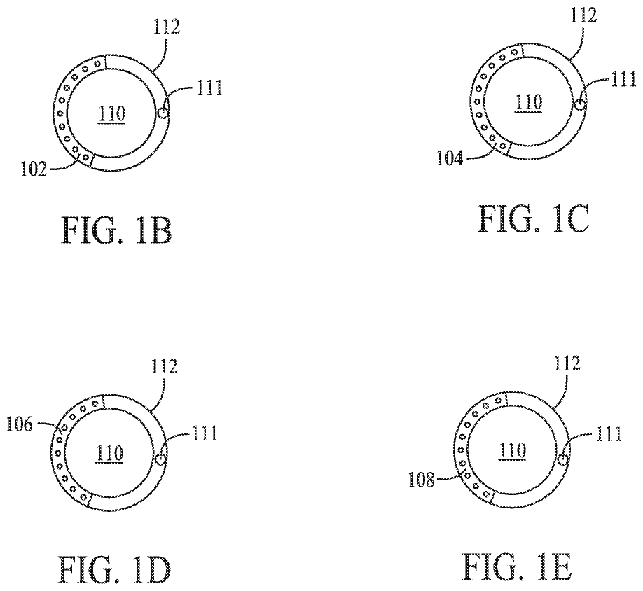

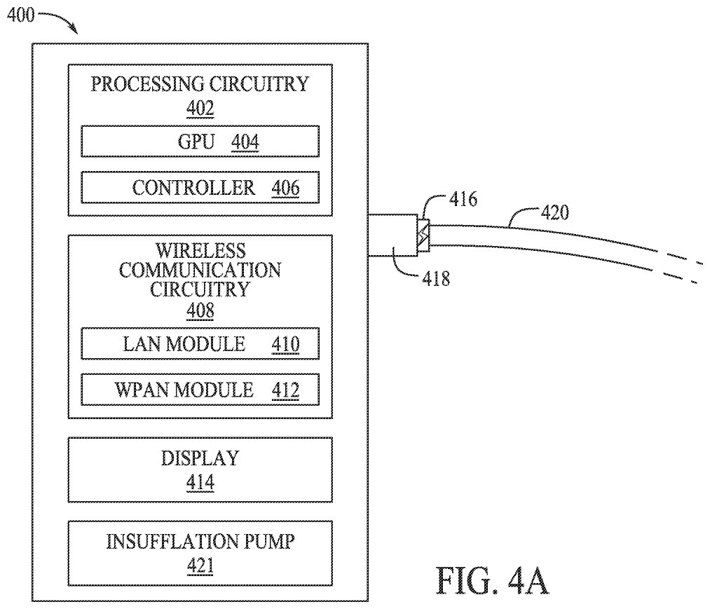

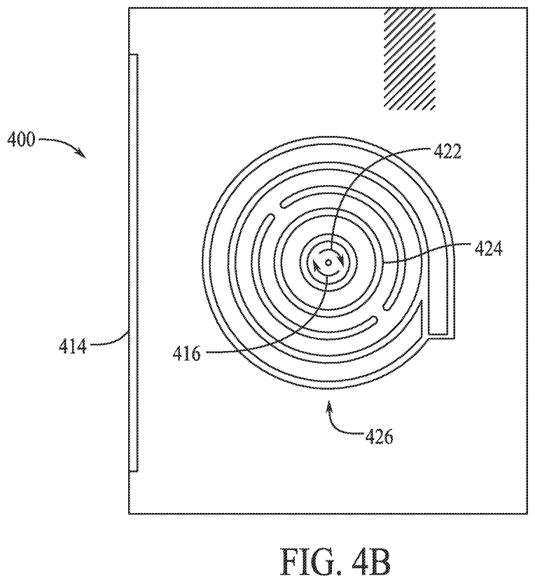

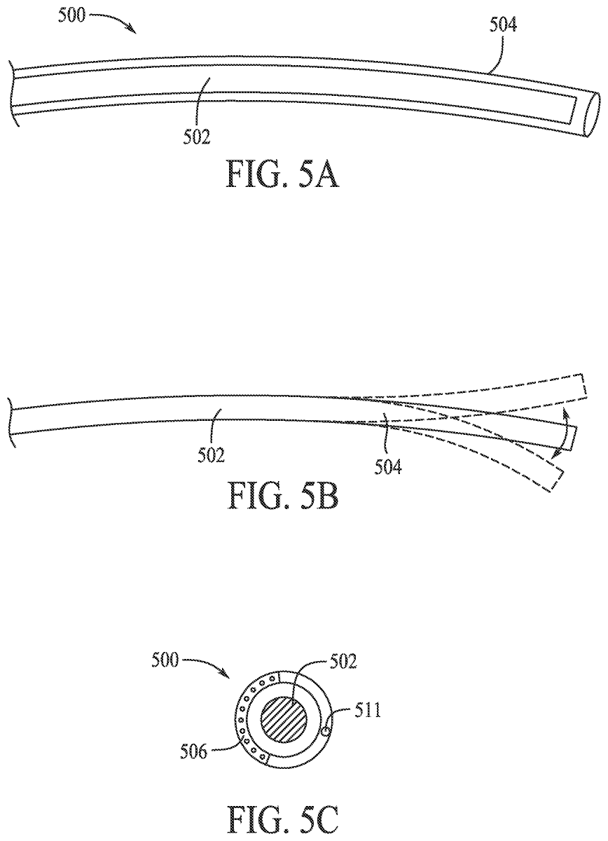

FIGS. 1A-1E show the distal end of an illustrative catheter tube 100, which may include a power source 102 (e.g., a battery), a spectrometer 104, a transceiver 106, a channel 111 that may or may not be used for insufflation of the gastro-intestinal tract during placement of the device, and an imaging device 108. While the example of FIGS. 1A-1E is provided in the context of components being are embedded a tube wall of the catheter tube 100, it should be understood that any of the power source 102, the spectrometer 104, the transceiver 106, and/or the imaging device 108 (collectively referred to here as “embedded components”) may additionally or alternatively be included in (e.g., embedded in a wall of) an articulated stylet (e.g., articulated stylet 420, 502, of FIGS. 4A, 4B, 5A-C) that may be inserted into a catheter tube such as the catheter tube 100, such that when the articulated stylet is fully inserted into the catheter tube the embedded components will be located at a distal end of the catheter tube.

As shown in FIG. 1A, the imaging device 108 may be positioned at the distal end of the catheter tube 100 closest to the tip, followed in order by the transceiver 106, the spectrometer 104, and the power source 102. The catheter tube 100 may be a hollow, substantially cylindrical tube made of polyurethane or silicone, for example. The catheter tube 100 may, for example, be formed from Pebax (or other polymers). In some embodiments, the catheter tube 100 may be a multilayer catheter that includes an inner liner (e.g., laser cure stainless steel, polyimide, FEP or PTFE), a jacket (e.g., Pebax, possibly of multiple durometers), and, optionally, a stainless steel braid or coil or both, depending on pushability, “steerability”, or flexibility requirements. The channel 111 may be coupled to a pump (e.g., an air or carbon dioxide pump), which may be housed in a remote device (e.g., the RCDC of FIGS. 4A, 4B). Air or other gases may be passed through the channel 111 by the pump to an opening in the end of the catheter tube 100 in order to perform insufflation of the gastro-intestinal tract of a subject, for example.

As shown in the cross-sectional view of FIG. 1B, the power source 102, including when the power source 102 is a battery, can be partially or completely embedded in or attached to a tube wall 112 of the catheter tube 100 (e.g., the power source 102 being encapsulated and isolated from the inner lumen of the catheter tube 100). The power source 102 may provide electric power to the spectrometer 104, the transceiver 106, and the imaging device 108. In some embodiments, the power source 102 may be located in the lumen 110 of the catheter tube 100. In some cases, with the power source 102 coupled to the catheter tube 100 and enclosed by the catheter tube 100, the power source 102 can be closer to each of the components that the power source 102 provides power to, which can prevent long electrical wires from being routed from each of the components (e.g., the imaging device 108, the transceiver 106, the spectrometer 104, etc.) to the power source 102 (e.g., when the power source 102 is positioned to be external to the catheter tube 100). In addition, the power source 102 being implemented as a battery can be advantageous in that the battery footprint, as opposed to other power sources 102, can be made advantageously small. For example, the power source 102 can be a lithium ion battery. As used herein a “lumen” refers to the central cavity of a catheter tube, with the lumen 110 referring to the central cavity of the catheter tube 100. As shown, the channel 111 may be embedded in the tube wall 112.

As shown in the cross-sectional view of FIG. 1C, the spectrometer 104 may be partially or completely embedded in or attached to a tube wall 112 of the catheter tube 100. In some alternate embodiments, the spectrometer may instead be disposed outside of the catheter tube 100, and connected to the distal end of the catheter tube 100 via an optical guide such as an optical fiber or bundle that is disposed in the lumen 110. The spectrometer may continuously analyze substances (e.g., biomarkers, such as those produced by organs of the human body) in the vicinity of the distal end of the catheter tube 100. For example, the spectrometer 104 may perform this analysis without directly interacting with sample substances being tested, instead leveraging the properties of light to perform spectral analysis. The results of an analysis performed by the spectrometer 104 may produce specific results related to biomarkers that may be included in a target organ, such as ion concentration, acidity, hormone levels, and toxicology analysis.

As shown in the cross-sectional view of FIG. 1D, the transceiver 106 may be partially or completely embedded in or attached to a tube wall 112 of the catheter tube 100. For example, the transceiver 106 may be a wireless personal area network (WPAN) transceiver that is configured to transmit and receive data wirelessly according to a WPAN protocol (e.g., Bluetooth® or Zigbee®). The transceiver 106 may wirelessly transmit data to a remote device (e.g., the RCDC device 400 of FIGS. 4A, 4B) for analysis. For example, data transmitted by the transceiver 106 may include a state of the distal end of the catheter tube 100 (e.g., position within an organ, proximity to surrounding structures), a state of an organ in which the catheter tube is located (e.g., volume of fluids, biomarker status, etc.). The transceiver 106 may also wirelessly transmit imaging data (e.g., topographic image data, still image data, and/or video data) captured by the imaging device 108 to the remote device. The transceiver 106 may transmit this data to the remote device both during the device placement process (e.g., as the catheter tube 100 is automatically driven to a target location), and during a continuous monitoring phase that may occur once the catheter tube 100 has reached the target location. In some embodiments, the transceiver 106 may only send data to the remote device without receiving instructions from the remote device. In other embodiments, the transceiver 106, in addition to receiving data to the remote device, may receive instructions from the remote device, such as instructions that, when executed, cause the devices in the catheter tip, such as the spectrometer 104, to perform specific functions (e.g., carrying out specific tests in the example of the spectrometer 104).

In some configurations, the transceiver 106 can include a single antenna configured to transmit and receive wireless signals therefrom, while in other cases, the transceiver 106 can include at least two separate antennas, in which a first antenna can be configured to receive wireless signals therefrom, and a second antenna can be configured to transmit wireless signals therefrom. In some non-limiting examples, the catheter tube 100, rather than (or in addition to) having the transceiver 106, can include a transmitter, in which the transmitter can transmit wireless signals therefrom, to, for example, the remote device.

As shown in the cross-sectional view of FIG. 1E, the imaging device 108 may be partially or completely embedded in or attached to a tube wall 112 of the catheter tube 100. The imaging device 108 may include one or more image sensors, which may be, for example, topographic image sensors and/or visual image sensors. The imaging device 108 may capture image data (“captured image data”) corresponding to structures (e.g., of the organ being traversed by the catheter tube 100) surrounding the distal end of the catheter tube 100. For example, LiDAR, time of flight imaging, visual image sensing (e.g., which may involve the capture of still images and/or video), or other applicable imaging techniques may be applied to capture the image data. Topographic image data that may be included in the captured image data may provide information related to the shape, volume, consistency, and location of the organ, or the portion of the organ, through which the distal end of the catheter tube 100 is traversing. The captured image data may be transmitted to and used by one or more artificial intelligence (AI) models executed by the remote device that is in wireless electronic communication with the transceiver 106, providing feedback to the AI model(s) regarding the location and position of the catheter tube 100 in the subject's body (e.g., in an organ thereof). Thus, the image data generated by the imaging device 108 may be used to guide the placement of the catheter tube 100 and to continuously monitor the location of the catheter tube 100 once it has reached the target location (e.g., to ensure the catheter tube 100 is not drifting away from the target location). By performing insufflation via the channel 111, visualization (e.g., as represented in the captured image data generated by the imaging device 108) of internal structures (e.g., gastro-intestinal structures) of the subject into which the catheter tube 100 is inserted may be improved. This improved visualization may also improve the recognition of landmarks that may be achieved by the AI model(s). Topographic and/or visual (e.g., two dimensional) image data acquired by the imaging device 108 as the catheter tube 100 and the articulated stylus maneuver through the enteral system may be saved in a computer memory of the remote device for simultaneous interpretation by AI models/algorithms or for later interpretation of the captured image data by qualified personnel for identification of abnormal tissue in the enteral cavity.

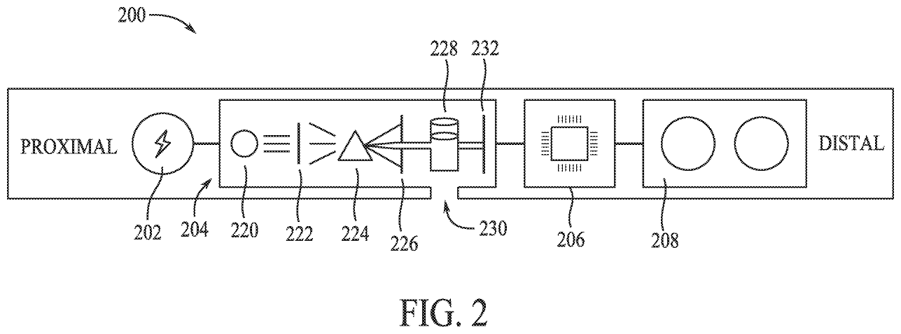

FIG. 2 shows an illustrative block diagram of devices and components within a distal end of a catheter tube 200 (e.g., which may correspond to the catheter tube 100 of FIG. 1A). The catheter tube 200 may include a power source 202 (e.g., corresponding to the power source 102 of FIG. 1A), a spectrometer 204 (e.g., corresponding to the spectrometer 204 of FIG. 1A), a transceiver 206 (e.g., corresponding to the transceiver 106 of FIG. 1A), and an imaging device 208 (e.g., corresponding to the imaging device 108 of FIG. 1A). In some embodiments, the spectrometer 204 may be a spectrophotometer. The spectrometer 204 may include a light source 220, a collimator 222, a monochromator 224, an exit slit 226, a sampling module 228, an access point 230, and a detector 232. The light source 220 may include one or more light emitting diodes (LEDs). The collimator 222 may include a lens that focuses the light generated by the light source 220 onto the monochromator 224 (e.g., through an entrance slit thereof, not shown). The monochromator 224 may include a prism (e.g., such as a Bunsen prism monochromator) or other optical device that transmits, to the sampling module 228 through the exit slit 226, a mechanically selectable narrow band of wavelengths of wavelengths of light received from the collimator 222. The size of the exit slit 226 may affect the wavelength(s) of light that may be output through the exit slit 226. Light (e.g., light output through the exit slit 226) passing through sampled material in the sampling module 228 be received by the detector 232. For example, the detector 232 may be a photodetector that measures the magnitude of the light that is able to pass through the sample, from which the absorbance and/or percent transmittance of the sample for the wavelength of the light may be determined.

Based on the absorbance and/or percent transmittance of the sample determined from the magnitude of light detected by the detector 232, the chemical make-up of the sample may be identified. For example, identification of the sample may be based on known spectroscopy properties of a compound (e.g., the sample) being studied. For example, the spectral wavelength of the compound may be determined, and using algorithms or models located in the RCDC, or in the cloud may be applied to identify the compound based on the spectral wavelength. For example, biomarkers that may be sampled and identified using the spectrometer 204 may include, but are not limited to, sodium, potassium, osmolarity, pH, medications, illicit drugs, digestive enzymes, lipids, fatty acids, blood, blood products, biomarkers for gastric cancer and/or gastric inflammation, biomarkers for intestinal cancer and/or intestinal inflammation, gastric proteome, and/or intestinal proteome.

In some embodiments, analysis to determine the identity of a substance sampled by the spectrometer 204 may be performed by a processor of a remote computing device (e.g., the GPU 404 of the device 400 of FIGS. 4A and 4B). In some embodiments, this analysis may be performed using one or more processors of a cloud computing environment.

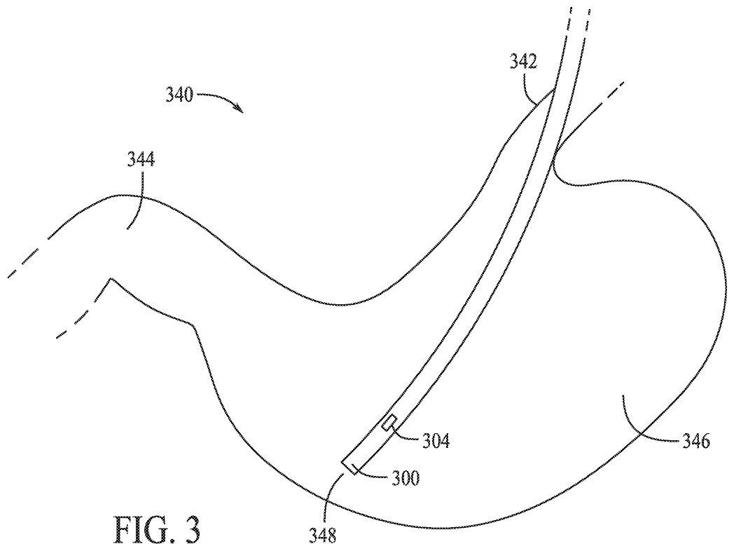

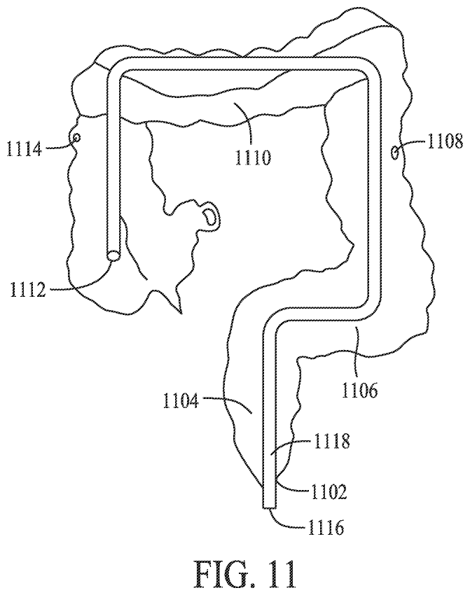

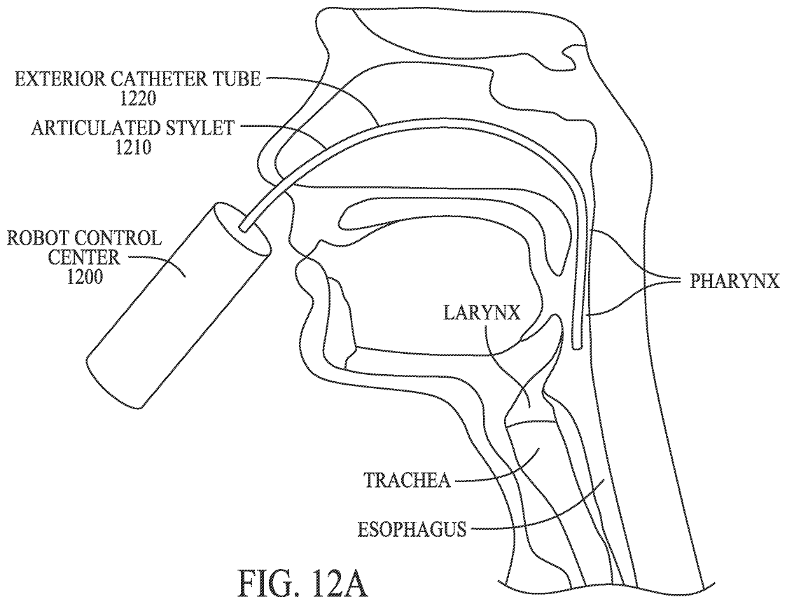

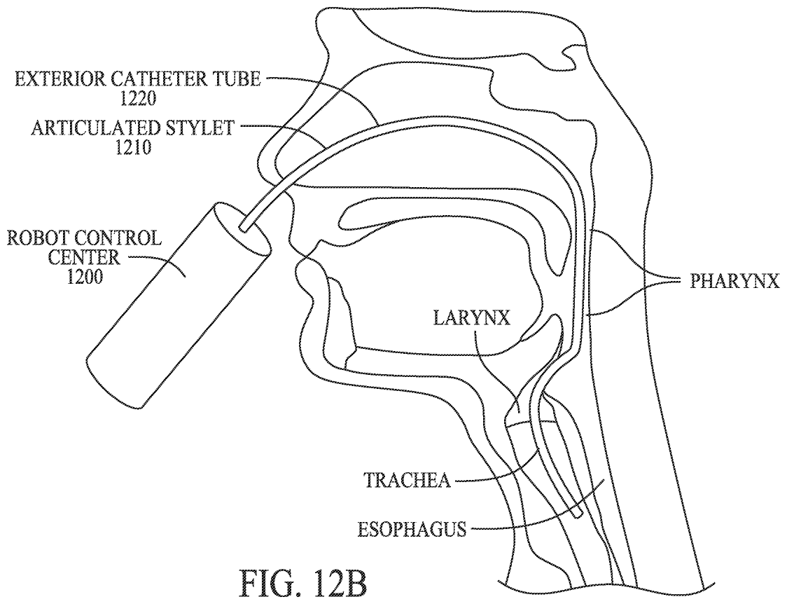

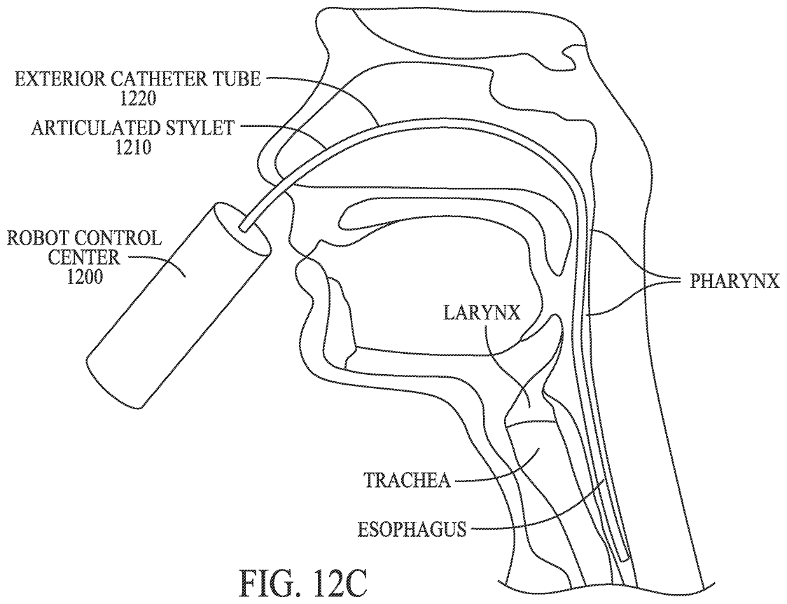

FIGS. 3 and 12C show examples of the placement of a catheter tube 300 (e.g., corresponding to catheter tube 100 of FIG. 1A or catheter tube 200 of FIG. 2) within an enteral system 346 of a subject (e.g., within the subject's gastrointestinal tract). As shown, the enteral system 346 may include a stomach 346, duodenum 344, and an esophagus 342. The catheter tube 300 may be driven (e.g., automatically, without manual guidance from a clinician) through the esophagus 342 to a target location at the pyloric antrum 348 of the stomach 346. In some embodiments, the catheter tube 300 may be driven to other target locations within the enteral system 346, such as the duodenum 344, other parts of the stomach 346, or other parts of the small or large intestine of the subject. Likewise, the area of access to the enteral system may be through the rectal region for placement and analysis for the lower enteral tract, as shown in FIG. 11, or in the pulmonary tract as demonstrated in FIG. 12B.

Once the tip of the catheter tube 300 has reached the target location one or more procedures may be performed using the catheter tube 300. For example, external content (e.g., medication, enteral feedings, or other biologically or chemically active substances, respiratory support, ventilation) may be delivered to the target location through the catheter tube, intestinal (including large bowel) content or stomach content may be removed (e.g., biopsied), and/or biomarkers (e.g., physical and/or biochemical biomarkers) may be continuously sampled using a spectrometer (e.g., spectrometer 104, 204 of FIGS. 1A, 1C, 2) disposed in the distal end of the catheter tube 300. In addition, all enteric/tracheal imaging data obtained during the placement of the catheter tube 300 in the enteral tract may be stored and analyzed (e.g., simultaneously analyzed) by one or more AI models/algorithms or subsequently by qualified personnel for identification of abnormal tissue in the enteral/tracheal cavity.

FIGS. 4A and 4B show a device 400 (sometimes referred to herein as a robotic control and display center (RCDC) 400) for the robotic control of an articulated stylet that may be inserted into the lumen (e.g., lumen 110 of FIGS. 1B-1E) of a catheter tube (e.g., catheter tubes 100, 200 of FIGS. 1A, 2) to automatically drive the distal end of the catheter tube to a target location within the body of a subject. The device 400 may include processing circuitry 402, wireless communication circuitry 408, a display 414, a 420, an insufflation pump 421, a screw-based lock mechanism 416, a wire motor control 418, a robotic control engine 424, a thread drive 422, and a loading dock 426.

The processing circuitry 402 may include a graphics processing unit (GPU) 404 and a controller 406 (e.g., which may include one or more computer processors). The processing circuitry may execute computer-readable instructions stored on one or more memory devices (not shown) included in (e.g., as local storage devices) or coupled to (e.g., as cloud storage devices) the device 400. For example, executing the computer-readable instructions may cause the processor to implement one or more AI models. These AI models may include, for example, one or more trained machine learning models, such as decision tree models, naïve Bayes classification models, ordinary least squares regression models, logistic regression models, support vector machine models, ensemble method models, clustering models (e.g., including neural networks), principal component analysis models, singular value decomposition models, and independent component analysis models.

For example, a neural network may be implemented by the processing circuitry 402 that receives a target location within the enteric cavity of a subject along with a stream of images (e.g., enteral/tracheal images captured/generated by the imaging device 108 of FIG. 1 and sent to the device 400 via the wireless communication circuitry 408) of features (e.g., organ tissue) around a catheter tube (e.g., catheter tube 100, 200 of FIGS. 1A, 2), and that outputs instructions that control the robotic control engine 424 and the articulated stylet 420 based on the received images, causing the articulated stylet 420 to move through the enteric cavity or respiratory tract of the subject until the catheter tube reaches the target location. The neural network may, for example, be trained and verified using 3D models of human enteric/respiratory pathways and anatomic images of naso/oral/rectal enteric and tracheal trajectories corresponding to catheter tube placement in multiple (e.g., thousands) of human subjects.

In some embodiments, the processing circuitry 402 may execute a volume sensing module configured to obtain volume measurements of an enteral space into which the catheter tube has been inserted. The volume measurements may be calculated based on three-dimensional volumetric data generated/acquired using one or more imaging techniques such as hyperspectral imaging, time of flight imaging using multiple wavelengths of light, and stereo imaging. The volume sensing module may, based on the volume measurements, determine a first volume value corresponding to a total volume of the enteral space, a second volume value corresponding to a first portion of the total volume that is empty, and a third volume value corresponding to a second portion of the total volume that is filled with material. The third volume may be calculated by subtracting the second volume from the first volume.

For example, the artificial intelligence (AI) based detection and tracking model which enables the RCDC to traverse autonomously may use a deep-learning detector, which may include both a deep-learning YOLO-based detector and convolutional neural network (CNN), to detect the nasal, oral, and rectal orifices, and the enteral/respiratory cavities, by further distinguishing between visually similar structures in the proximal environment. For enteral/tracheal spatial detection, a 26-layer or greater CNN based detection model may be employed. In such a model, the first 24 layers may be fully convolutional layer that are pre-trained on Imagenet dataset, and the final two layers may be fully connected layers which output the detected tissue/organ. For tracking, a median-flow filtering based visual tracking technique to predict the motion vector for the robotic placement device may be employed, using estimations of the location of an object with sparse optical flow. The tracking based system may be based on the assumption that an object consists of small and rigidly connected blocks or parts which more synchronously together with motion of the whole object, such as the enteral cavity or respiratory tract.

For example, the AI model initialization may be achieved by establishing a bounding box in which the nasal/oral/rectal orifice or enteral cavity is located at first, and within this region of interest a sparse grid of points may be generated. The motion of the enteral structure detected by optical flow in the captured images may be computed as the median value of differences between coordinates of respective points that are in the current and preceding images. Only those points which have been regarded as reliable during the filtering may be considered, such that the algorithm may estimate the object scale variations.

For example, the AI model implementation and enteral/tracheal object detection may be accomplished via YOLO-based algorithm and object tracking that may be accomplished via median flow tracker, as implemented through Python. The environment may be built on Ubuntu. The graphics processing unit (GPU) integration cuDNN and CUDA toolkit may be used. The training segment may be implemented by supplying annotated images to Keras implementation of YOLO. The Keras and TensorFlow backend may be used, and the dataset may be created with annotated software VoTT (Microsoft, Redmond, WA), with an adopted learning rate of 10³ for 1,000 training epochs and saved model parameters every 100 epochs. The detection segment may again be implemented based on Keras running TensorFlow on the backend. For tracking, the tracking API in OpenCV may be used. The bounding box may be detected by YOLO and passed to Median Flow tracker at m:n ratio, in order to realize real-time detection and tracking.

In some embodiments, rather than being stored and executed by the processing circuitry 402, the computer-readable instructions corresponding to the AI models may be stored and executed by cloud-based memory devices and computer processors. Data (e.g., image and spectrometer data) taken as inputs by the AI models may be sent to such cloud-based memory devices and computer processors by the device 400 via one or more communication networks using the wireless communication circuitry 408. The wireless communication circuitry 408 may additionally receive the outputs of these AI models after they have processed the data. In this way, the requirements for the processing capabilities of the local processing circuitry 402 of the device 400 may be less than if the AI models needed to be executed locally, which may generally decrease the cost and, in some cases, the footprint of the device 400. However, such cloud-based solutions generally require network (e.g., internet) connectivity and may take longer to execute the AI models than local hardware (e.g., in cases where cloud and local processing capabilities are assumed to be equal). In some embodiment, AI models may be executed to perform data analysis by both the local processing circuitry 402 and cloud-based processors (e.g., such that biomarker analysis is performed locally and robotic driven navigation analysis is performed by cloud-based processors, or vice-versa).

The wireless communication circuitry 408 may include a local area network (LAN) module 410 and a wireless personal area network (WPAN) module 412. The LAN module 410 may communicatively couple the system 400 to a LAN via a wireless connection to a wireless router, switch, or hub. For example, the LAN module 410 may communicate with one or more cloud computing resources (e.g., cloud computing servers) via network connections between the LAN and an external network to which the cloud computing resources are connected (e.g., over a wide area network (WAN) such as the internet). The WPAN module 412 may communicate with a transceiver (e.g., transceiver 106, 206 of FIGS. 1A, 1D, 2) disposed at a distal end of the catheter tube using one or more antennas according to a short-wavelength UHF wireless technology standard (e.g., Bluetooth®) via an established WPAN. For example, the WPAN module 412 may receive image data (e.g., 3D topographic image data) generated by an imaging device (e.g., imaging device 108, 208 of FIGS. 1A, 1E, 2) disposed in the distal end of the catheter tube, which may be a topographic imaging device. The image data may be transmitted to the WPAN module 412 in real-time as the catheter tube is driven toward the target location in the subject's enteric cavity or respiratory tract. The WPAN module 412 may also receive spectrometer data from a spectrometer (e.g., spectrometer 104, 204 of FIGS. 1A, 1C, 2). The WPAN module 412 may provide this spectrometer data to the processing circuitry 402, which may analyze the spectrometer data to identify one or more chemical substances sampled by the spectrometer (e.g., using one or more of the AI models trained to perform such analysis).

In some embodiments, rather than using the WPAN module 412 to communicate with the communication circuitry (e.g., transceiver) disposed in the distal end of the catheter tube, a direct wired connection to the communication circuitry or the LAN module 410 may be used to transfer data to and from the communication circuitry of the catheter tube.

The articulated stylet 420 (sometimes referred to herein as a “robotic navigating articulated stylet”) may be inserted into the lumen (e.g., lumen 110 of FIGS. 1B-1E) of a catheter tube (e.g., catheter tube 100, 200 of FIGS. 1A, 2). The articulated stylet 420 may be locked to a port of the device 400 using a screw-based lock mechanism 416. The screw-based lock mechanism 416 can work in several different ways. In one embodiment, the screw-based lock mechanism 416 may operate similarly to a Tuohy Borst valve or a standard coaxial articulated stylet lock that screws down a silicone or elastomer ring that would compress around the articulated stylet 420 to lock it in place. In another embodiment, the screw-based lock mechanism 416 could include a screw/wedge aligned substantially perpendicularly to the length of the articulated stylet 420, which may to “pinch” the articulated stylet 420 as the screw/wedge is rotated into a “locked” state, locking the articulated stylet 420 in place. In yet another embodiment, the screw-based lock mechanism 416 may include a colt mechanism that includes a coaxial screw mechanism that operates by bringing several metal (or plastic) fingers in around the articulated stylet 420 as the mechanism is rotated to lock the articulated stylet 420 in place (e.g., similar to a drill bit chuck). In some embodiments, the articulated stylet 420 may be embedded in the catheter tube itself, rather than being inserted into the lumen of the catheter tube. The articulated stylet 420 may be formed from one of a variety of applicable materials. In some embodiments, the articulated stylet 420 may be formed from stainless steel. For example, the articulated stylet 420 may include a single stainless steel filament or a braided or wound stainless steel cable. In some embodiments, the articulated stylet 420 may be made from Nitinol, which may provide shape memory or extreme flexibility characteristics. The articulated stylet 420 may have an articulated distal end (e.g., articulation 504 of FIG. 5A, 5B), which may be controlled by the robotic control engine 424, such that the articulated stylet 420 may change direction as it guides the catheter tube through the enteral cavity or respiratory tract of a subject. For example, the robotic control engine 424 may directly navigate the articulated stylet 420, and therefore the catheter tube, from an access site (e.g., the nose or mouth of the subject) through the subject's enteral cavity or respiratory tract until the distal end of the articulated stylet 424 and the catheter tube reaches its destination. The robotic control engine may control the direction of the articulated stylet 420 based on navigation data output by a navigation AI model of the AI models that may be executed by the processing circuitry 402 and/or cloud-based processing circuitry in communication with the wireless communication circuitry 408. In some embodiments, more than just the distal end of the articulated stylet 420 may be articulated, with additional articulations being included along some or all of the length of the articulated stylet 420. In this way, the articulated stylet 420, as controlled by the robotic control engine 424 may steer the catheter tube to a target location in the subject's enteral cavity or respiratory tract, while avoiding areas that may pose safety risks to the subject, such as the subject's larynx, vocal cords, and lower airway. The robotic control engine 424 may additionally apply a robotic feedback system once the catheter tube has reached its target location in the subject's enteric cavity or respiratory tract, the robotic feedback system maintaining the catheter tube at the target location (e.g., by continuously monitoring the position of the distal end of the catheter tube using one or more AI models based on topographic image data generated by an imaging device embedded in the catheter tube). Various mechanisms of control may be carried out by the robotic control engine 424 to control the movement of the articulated distal end of the articulated stylet. In some embodiments, the mechanism of control may include a pull-wire type configuration where multiple wires are attached at different points in the catheter tube around locations (e.g., articulation joints) where movement (e.g., bending/flexing) of the catheter tube is desired. These wires would be connected to spools in the motor mechanism of the robotic control engine 424. The spools may rotate in a first direction (e.g., clockwise or counterclockwise) to wind the wires up (flexing the catheter tube tip back toward its proximal end) and may rotate in a second direction (e.g., opposite the first direction) extend the wires (e.g., causing the catheter tube to stop flexing). Positioning these wires at “strategic” points along the longitudinal axis (e.g., length) of the catheter tube could allow steering of the catheter tube in nearly any direction. In another embodiment, of this mechanism of control could utilize a series of links disposed inside the catheter that could be addressed via multiple Nitinol wires that are coupled to the series of links. When a current is supplied to the Nitinol wires (e.g., supplied by a power supply controlled by the robotic control engine 424), the Nitinol wires shrink, flexing the catheter. Removal of the current causes Nitinol wires to relax, extending the catheter.

For advancement and retraction of the articulated stylet 420, a drive system (e.g., a drive rod, worm gear, or rack and pinion based drive system, depending on the accuracy required) may be included in the robotic control engine 424 that may be controlled to drive the articulated stylet forward and back (e.g., using a single motor). A transmission may be included in the robotic control engine 424, which may be used to enable automatic rotation and articulation of the catheter when the articulated stylet 420 is inserted, as well as the forward/reverse driving of the articulated stylet 420. The transmission would also enable steering.

A display 414, which may be an electronic display including an LCD, LED, or other applicable screen, may be included in the device 400. The display 414 may display the status information related to the articulated stylet, the catheter tube, the components of the catheter tube, and one or more organs of a subject that are proximal to the distal end of the catheter tube. For example, the displayed data may include information regarding placement of the catheter tube (e.g., the tip and/or distal end of the catheter tube), the status of the components of the catheter tube, and biomarkers detected by the spectrometer embedded in the distal end of the catheter tube. In some embodiments, some or all of the information shown on the display 414 may also be transmitted to other electronic devices by the LAN module 410 and subsequently displayed on such devices. For example, such electronic devices may include personal electronic devices phones and tablets of doctors and nurses, as well as computer systems having a monitors disposed at subjects' bedsides, any of which may be connected to the same LAN as the LAN module 410. Data transmitted to these devices may be stored as part of an Electronic Health Record for the corresponding subject, and may be incorporated in to Clinical Decision Support Systems (e.g., for use in patient management).

An insufflation pump 421 may be included in the RCDC 400, which may be an air pump, carbon dioxide pump, or any applicable pump configured to output a gas (e.g., a gas appropriate for use in insufflation). The insufflation pump 421 may be coupled to an insufflation channel (e.g., channel 111, 511 of FIGS. 1A-1E, 5C), through which insufflation of an interior cavity of a subject (e.g., a gastro-intestinal tract of the subject) may be performed.

The thread drive 422 may control the extension and retraction of the articulated stylet 420, according to navigation data output by the navigation AI models described previously.

The loading dock 426 may store the portion of the guide-wire that is not in use. The articulated stylet 42.0 may be longer than the catheter to be placed, such that the catheter tube can be driven forward fully without utilizing the full length of the articulated stylet 420. In some embodiments, the articulated stylet 420 may run on a spool or through a linear tube of the loading dock 426, depending on the application and/or the drive mechanism. In some embodiments, the articulated stylet 420 may be loaded and addressed by the thread drive 422 by feeding the articulated stylet tip into the drive gears/rod/rack of the thread drive 422. In some embodiments, the length of the articulated stylet 420 may be selected to accommodate having the thread drive 422 far enough from the patient to allow for the RCDC to be positioned at the side of the patient's bed. In such embodiments, fixation may be provided for the articulated stylet 420 at the patient's mouth (e.g., via a biteblock) in order to improve the mechanical drive of the articulated stylet 420.

FIG. 5A shows an illustrative placement of a, articulated stylet 502 (e.g., articulated stylet 420 of FIGS. 4A, 4B) in a catheter tube 500 (e.g., catheter tube 100, 200 of FIGS. 1A-1E, and 2) to form a catheter assembly. The articulated stylet 502 may include a distal end having an articulation 504, which may have three degrees of freedom by which it may navigate through three-dimensional space, which may include use of plunge, rotation, and deflection of the articulation 504. For example, the articulation 504 may be located around 10 cm away from the distal end of the articulated stylet 502. An illustrative placement of an insufflation channel 511 (e.g., channel 111 of FIG. 1) in a tube wall of the catheter tube 500 is shown.

FIG. 5B shows an illustrative range of motion of the distal end of the articulated stylet 502 as the articulation 504 is controlled by a robotic control engine (e.g., robotic control engine 424 of FIG. 4A) of an RCDC device (e.g., device 400 of FIG. 4A, 4B), such that the robotic control engine may navigate the articulated stylet 502, and thereby the catheter tube 500, to a target location within the enteral system or respiratory tract of a subject.

FIG. 5C shows a cross-section of a distal portion of the catheter tube 500 when the articulated stylet 502 is fully inserted into the catheter tube 500. As shown, when fully inserted, the articulated stylet 502 may overlap with one or more embedded components of the catheter tube, such as an imaging device (e.g., imaging device 108 of FIG. 1) embedded in a tube wall 506 of the catheter tube 500.

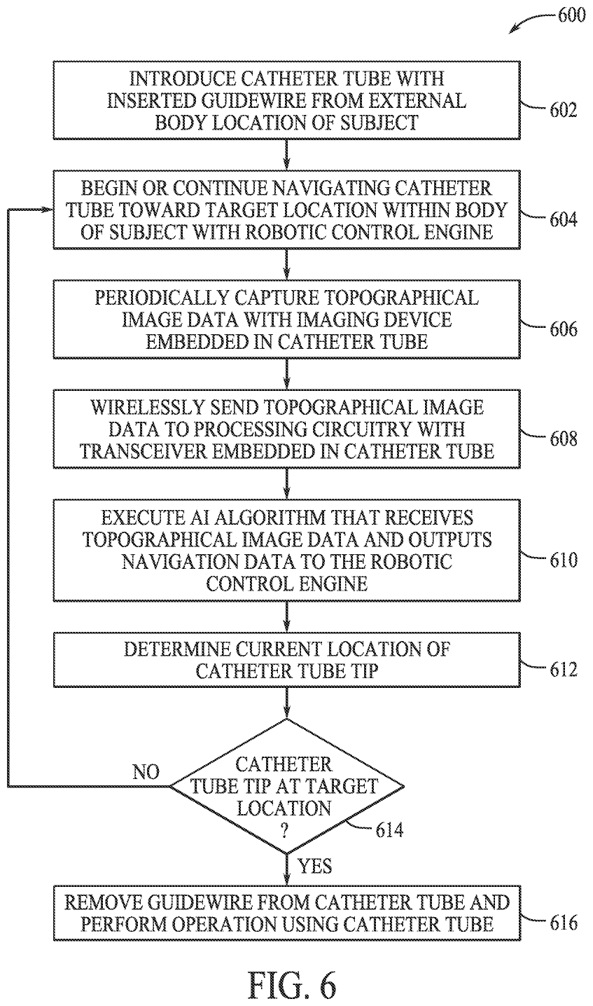

FIG. 6 shows an illustrative process flow for a method 600 by which one or more AI models may be implemented to navigate an articulated stylet (e.g., articulated stylet 420, 502 of FIGS. 4A, 5A-5C) and catheter tube (e.g., catheter tube 100, 200, 500 of FIGS. 1A, 2, 5A) to a target location within a subject's enteral system. For example, the method 600 may be performed by executing computer-readable instructions stored on one or more local storage devices of a computer system (e.g., device 400) or remote storage devices (e.g., cloud-based storage devices coupled to the computer system via an electronic communication network), using one or more computer processors (e.g., processing circuitry 402 of FIG. 4, or cloud-based processing circuitry coupled to the computer system via an electronic communication network).

At step 602, the catheter tube, with the articulated stylet fully inserted, is introduced from an external body location of a subject. For example, the external body location through which the catheter tube is introduced may be the subject's mouth, nose, rectum, or a surgical incision on the subject's body.

At step 604, the catheter tube may be navigated, by driving the articulated stylet, toward a target location within the subject's body (e.g., within an enteral cavity of the subject). The navigation of the catheter tube may be performed by extending the articulated stylet into the body of the subject and controlling the direction, rotation, and movement of at least an articulated distal end of the articulated stylet using a robotic control engine (e.g., robotic control engine 424 of FIGS. 4A, 4B).

At step 606, an imaging device (e.g., imaging device 108 of FIGS. 1A, 1E) included in the catheter tube may capture (e.g., continuously capture) image data, such as 3D topographic image data, time of flight image data, visual (e.g., 2D) image data, and/or other applicable enteric/tracheal image data, of structures (e.g., organ tissue) in proximity to the distal end of the catheter tube. The imaging device may send the captured image data to a transceiver included in the distal end of the catheter tube. The captured image data may be stored at a computer memory that is included in or communicatively coupled to the robotic control engine. Once stored, the captured image data may be analyzed simultaneously (e.g., in near-real time) by one or more AI algorithms/models or may be subsequently analyzed by qualified personnel to identify abnormal tissue in the enteral cavity.

At step 608, the transceiver may wirelessly transmit the captured image data to processing circuitry (e.g., processing circuitry 402 of FIG. 4A) of a computer system. This wireless transmission may be performed via communication between the transceiver and a WPAN module (e.g., WPAN module 412) of the computer system.

At step 610, the processing circuitry of the computer system may execute one or more AI models (e.g., navigation AI models that may include a neural network). The AI models may receive the captured image data as inputs and, after processing the captured image data through a neural network and/or median-flow filtering, may output navigation data to the robotic control engine. The navigation data may include instructions for how the robotic control engine should manipulate, articulate, rotate, and/or drive the articulated stylet toward the target location, and may further include information defining a position of the catheter tube in the enteral cavity or respiratory tract of the subject.

At step 612, the processing circuitry may determine the current location of the catheter tube tip based on the navigation data. The processing circuitry may further determine whether the current location of the catheter tube tip corresponds to the target location.

At step 614, if the current location of the catheter tube tip is determined to correspond to the target location, the method 600 proceeds to step 616. Otherwise, the method 600 returns to step 604, and the robotic control engine continues to navigate the catheter tube and articulated stylet based on the navigation output by the AI models.

At step 616, the articulated stylet is removed from the catheter tube, and an operation is performed using the catheter tube. For example, substances (e.g., nutritive substances, medicine, or ventilation) may be delivered to the target location of the subject's enteral cavity or respiratory tract through a lumen of the catheter tube. Alternatively, substances (e.g., biopsied tissue or fluids) at the target location of the subject's enteral cavity may be retrieved through the lumen of the catheter tube.

In some embodiments, the catheter tube may remain indwelling in the patient for a standard duration of time following step 616, as clinically indicated. The indwelling catheter tube may be used for continuously monitoring, continuously sampling, providing food, delivering medicine, or providing airway support.

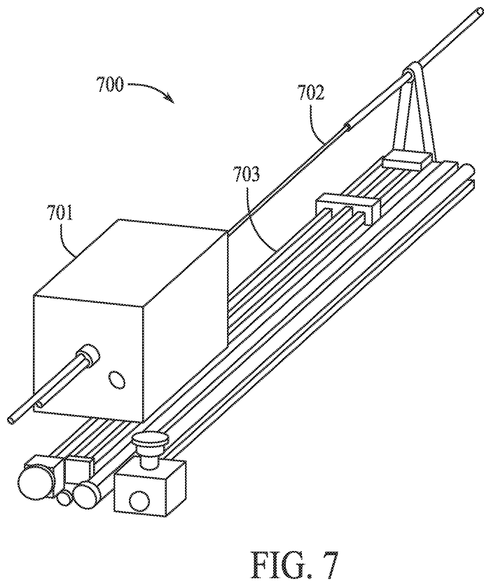

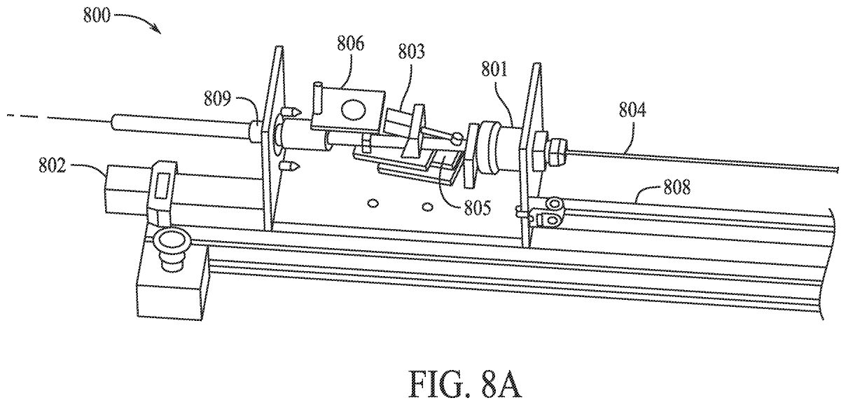

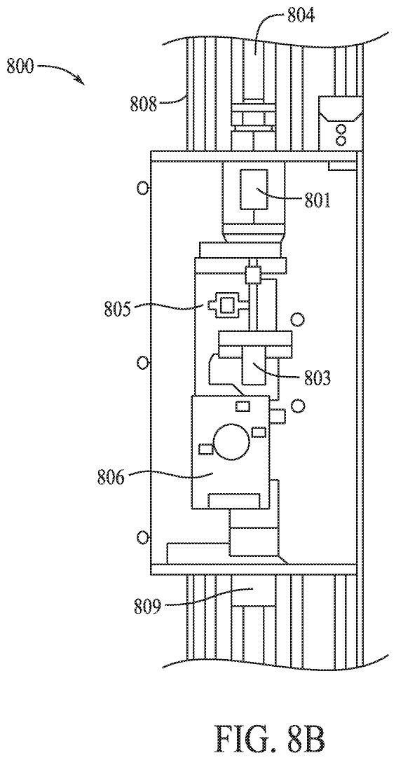

FIG. 7 shows a potential embodiment of an RCDC (e.g., which may correspond in whole or in part to the RCDC 400 of FIGS. 4A and 4B). The example RCDC 700 may include a carriage 701, an articulated stylet 702, and a slide 703. The carriage 701 may house imaging devices and motors that may move with the catheter into which the articulated stylet 702 is inserted. The articulate stylet 702 may be mounted to the carriage 701. The carriage 701 moves along the slide 703 to control the position of the tip of the articulated stylet 702. A controllable motor (e.g., linear slide motor 802, FIG. 8A) may drive the movement of the carriage 701 along the slide 703, For example, the controllable motor may move the carriage 701 along the slide 703 according to instructions received by the controllable motor from a computer processor (e.g., processing circuitry 402, FIG. 4A) of the RCDC 700.

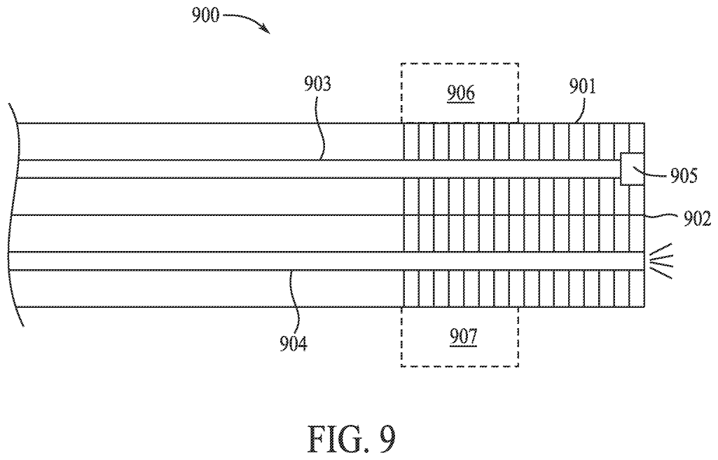

FIGS. 8A and 8B show perspective and top-down views, respectively, of the interior details of the carriage 701 of FIG. 7. The example carriage interior 800 contains motors to control the rotation and tip actuation of the articulated stylet 804 (e.g., articulated stylet 702, FIG. 7). The motors of the present example may allow control of the articulated stylet 804 with 3 degrees of freedom which would be the least number needed to navigate the tract. More degrees of freedom may be added to increase the number of articulated joints or change the contour or shape as the articulated stylet 804 is navigated/driven to a target location. The rotation of the articulated stylet 804 is controlled by a rotation motor 801, while the actuation of the tip of the articulated stylet 804 is controlled by a pull wire motor 803. The carriage (e.g., carriage 701, FIG. 7) moves along the linear slide 808 (e.g., slide 703, FIG. 7) controlled by a linear slide motor 802. The rotational motor 801 may also rotate a center shaft inside the carriage. A camera 805 and a light source 806 may be mounted on the center shaft. The camera 805 and the light source 806 may rotate with the articulated stylet 804 to allow for implantation of an image guide (e.g., image guide 903, FIG. 9) and light pipe (e.g., light pipe 904, FIG. 9). The camera 805 and light source 806 may utilize a variety of wavelengths of illumination, or imaging methodologies, including topographical imaging, time of flight imaging, and/or still and/or video visual (e.g., 2D) imaging. The shaft 809 may be capable of continuous 360 degree rotation without binding any of the necessary wiring to power or communication with the camera 805 or light source 806.

FIG. 9 shows a lateral cross section of an exemplary articulated stylet 900. The articulated stylet 900 has a bending section 901 at its distal tip. The bending section is attached to a pull wire 902 that can be actuated by a pull wire motor (e.g., pull wire motor 803, FIGS. 8A and 8B). The Enhanced Articulated Stylet may also include an image guide 903 with lensing 905. The image guide 903 provides a pathway by which optical data received through the lensing 905 may be passed back to the camera (e.g., camera 805, FIGS. 8A and 8B) within a carriage (e.g., carriage 701, carriage interior 800, FIGS. 7, 8A, and 8B). Illumination for the camera is provided through the light pipe 904, which connects back to a light source (e.g., light source 806, FIGS. 8A and 8B) in the carriage. In some embodiments, the image guide 903 and a light bundle that includes the light pipe 904 may additionally or alternatively be embedded within a catheter tube (e.g., a catheter tube 100, 200, 500 of FIGS. 1A, 2, 5A) external to the articulated stylet 900 (e.g., into which the articulated stylet 900 is inserted). In some embodiments, the articulated stylet 900 may include a stylet spectrometer 906 and/or a stylet transceiver 907 at or near the distal end (FIG. 9).

A potential embodiment of the RCDC 700 of FIG. 7 utilizes the continuous rotation mechanism 809 of FIGS. 8A and 8B to wire local components held within the carriage 701 back to a centralized processing and/or display unit (e.g., display 414, FIG. 4A).

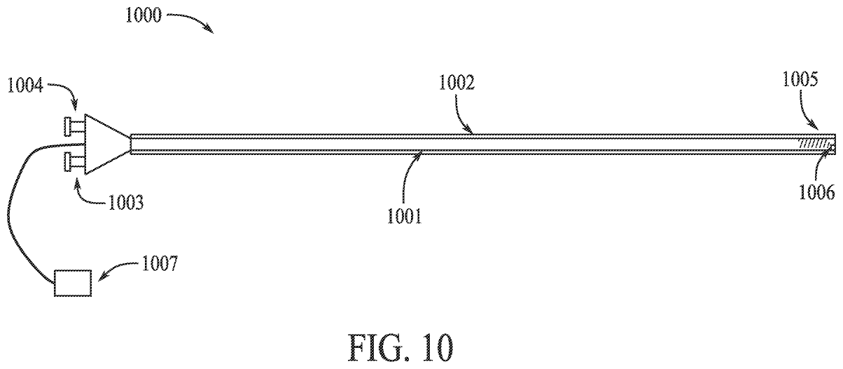

FIG. 10 shows an exemplary assembly 1000 that includes an articulated stylet 1001 that has been inserted into a catheter tube 1002. The articulated stylet 1001 contains a distal bending section 1005, and an optical window 1006. The optical window may connect to an image guide (e.g., image guide 903, FIG. 9), and connect back to a RCDC (e.g., RCDC 400, 700, FIGS. 4A, 4B, and 7) via the optical connector 1007. In some embodiments, optical components may additionally or alternatively be embedded within the wall of the catheter tube 1000 (e.g., catheter tube 100, 200, 500, 1002, of FIGS. 1A, 2, 5A, 10). The catheter tube 1002 has the articulated stylet 1001 inserted into it, and may remain indwelling within the enteral cavity or respiratory tract of the patient after the articulated stylet 1001 is removed. The articulated stylet 1001 may also include a proximal insufflation port 1003 and proximal enteral access port 1004. Both of the ports 1003 and 1004 provide the RCDC access to an embedded lumen, which may be embedded within either the articulated stylet 1001 or the catheter tube 1002.

FIG. 11 shows an illustrative placement of an articulated stylet 1118 (e.g., articulated stylet 420, 502, 702, 804, 900, 1001, FIGS. 4A, 5A, 5B, 7, 8A, 8B, 9, 10) within the colon of a subject. As shown, the articulated stylet 1118 may enter the anus 1102, pass through the rectum 1104, pass through the sigmoid colon 1106, pass thorough the descending colon 1108, pass through the transverse colon 1110, and pass through the ascending colon 1114 to reach a target location. As shown, transitions from the rectum 1104, to the sigmoid colon 1106, from the sigmoid colon 1106 to the descending colon 1108, from the descending colon 1108 to the transverse colon 1110, and from the transverse colon 1110 to the ascending colon 1114 may be made via articulations of the stylet 1118. The distal end 1112 of the articulated stylet 1118 may be located at the target location, and may include an imaging device (e.g., imaging device 108, 208, FIGS. 1A, 1E, AND 2). The imaging device may capture image data (e.g., topographic, time of flight, and/or visual image data) of structures, contents, interior walls, and/or the like of or within the colon as the articulated stylet 1118 traverses the colon, and the image data may be stored in a computer memory device of or communicatively coupled to a robotic control device (e.g., RCDC 400, 700, FIGS. 4A, 4B, 7). The captured image data may be analyzed in real or near-real time as the articulated stylet 1118 traverses the colon to the target location. The robotic control device may be coupled to a proximal end 1116 of the articulated stylet 1118, and may control the plunge, rotation, tip deflection, and advancement/retraction of the articulated stylet 1118.

The catheter tube and RCDC described above may have a variety of practical applications.

In one example application, the catheter tube and RCDC may be applied together for automated gastro-intestinal tract in vivo direct catheter tube navigation for identification, imaging and potential sampling of abnormal tissue samples.

In another example application, the catheter tube and RCDC may be applied together for automated gastro-intestinal tract in vivo direct catheter tube navigation for surveillance of abnormal tissue samples.

In another example application, the catheter tube and RCDC may be applied together for automated lower intestinal tract in vivo direct catheter tube navigation for surveillance of abnormal tissue samples, obtaining topographic or visual image data to be stored at a computer memory of or communicatively coupled to the RCDC, which can be analyzed simultaneously by one or more AI models/algorithms or subsequently by qualified personnel for identification of abnormal tissue in the enteral cavity.

In another example application, the catheter tube and RCDC may be applied together for automated gastro-intestinal tract in vivo direct catheter tube navigation for sampling of biomarkers for gastro-intestinal cancer, inflammatory disease, and malabsorption syndromes.

In another example application, the catheter tube and RCDC may be applied together for automated gastro-intestinal tract in vivo direct catheter tube navigation for assistance in operative procedures including percutaneous feeding access, and/or various laparoscopic interventions including endoscopic bariatric surgery, and endoscopic surgery of biliary tracts.

In another example application, the catheter tube and RCDC may be applied together for automated respiratory tract in vivo direct endotracheal intubation tube navigation for automated intubation.

In another example application, the catheter tube and RCDC may be applied together for automated respiratory tract in vivo direct endotracheal intubation tube navigation for ventilatory support.

In this embodiment, the use of an automated, autonomous, mobile robot for endotracheal intubation could be critical. This can be accomplished using visual based data and advanced data analytics and artificial intelligence as disclosed herein to drive the device that allows for early, safe, and dependable endotracheal intubation.

In this embodiment the stylet of the robot extending from the RCDC would be placed through one end of the endotracheal tube to be inserted and brought out the other end. The stylet would then be placed either in the nostril of the patient (either the left or right nostril) or in the mouth of a patient (alongside a standard oral-pharyngeal tube). The robot would at this point start its process. Using images obtained from the visual and topographic cameras at the tip of the stylet, the computer's algorithm would begin to recognize structure in the nasopharynx or oropharynx (depending on the site of insertion) and given these images the robot would direct the stylet down the pharynx into the larynx. At this point the epiglottis will come into the sight of the robot, which will be recognized. The algorithm will recognize the juncture of the larynx anteriorly and the esophagus posteriorly and through the use of the actuators and motors that control all of its degrees of freedom, steer the stylet anteriorly through the larynx and through the vocal chords into the trachea. This will all be done using computer vision as a guide, without input required from any clinician at the patient's side. The decisions guiding the direction of the stylet will all be automated through the computer algorithm and controlled through the mechanical system of the device.

Once in the trachea, the device will provide images of the inside of the trachea. It will be able to give confirmatory evidence of the correct placement of the stylet in the trachea, through the vocal cords and above the level of the division of the trachea into mainstem bronchi, known as the carina. This is critical as it will confirm the position of the stylet through identification of the vocal cords therefore ensuring a secure airway, but will not be placed so deep as to create intubation of one of the bronchi that could cause ventilation of only one lung.

In one embodiment, this confirmation could be provided as a live photograph to the clinicians at the patient's side or a three-dimensional topographic reconstruction.

In one embodiment, placement of the endotracheal stylet can be confirmed to be in the airway by the use of a stylet spectrometer 906 (FIG. 9) similar to the previously described spectrometer which is attached to a distal end of the stylet along with a stylet transceiver 907 (FIG. 9) and which can be used to detect carbon dioxide in the specimen being sampled. The detection of carbon dioxide provides an indication that the stylet is in the airway as opposed to in the esophagus, where no carbon dioxide would be expected to be detected. This would be a second form of confirmation of proper placement (along with identification of the vocal cords).

The correct placement of the endotracheal tube is critical as an incorrectly placed endotracheal tube is a major complication that can expose patients to a prolonged period with low oxygenation and tissue ischemia.

Once the stylet has been confirmed to be in the correct location, both through the use of visual images or three dimensional image reconstructions, as well as through the use of spectroscopic identification of intraluminal carbon dioxide, the endotracheal tube which is placed over the outside of the robotic stylet will simply be advanced over the stylet into the correct placement in the patient trachea.

FIG. 12A shows a diagram of the robotic control center 1200 advancing the catheter device with its inner articulated stylet 1210 and the exterior catheter tube 1220, demonstrating the self-driving robot advancing to the inflection point of the larynx where the robot can turn anteriorly into the larynx and trachea or posteriorly into the esophagus. At this point, the computer algorithm, basing its decisions on the images obtained from the cameras on the robot's stylet, will guide the mechanical part of the robotic control to turn the stylet anteriorly in the pharynx into the larynx, through the vocal cords and into the trachea.

FIG. 12B shows a diagram of the robot advancing the catheter device with the inner articulated stylet, and the exterior catheter tube, demonstrating the self-driving robot advancing beyond the inflection point of the pharynx having driven anteriorly into the larynx and trachea for pulmonary access.

FIG. 12C shows a diagram of the robot advancing the catheter device with its inner articulated stylet, and the exterior catheter tube, demonstrating the self-driving robot advancing to the point beyond the inflection point of the pharynx having driven posteriorly into the esophagus for upper enteral access.

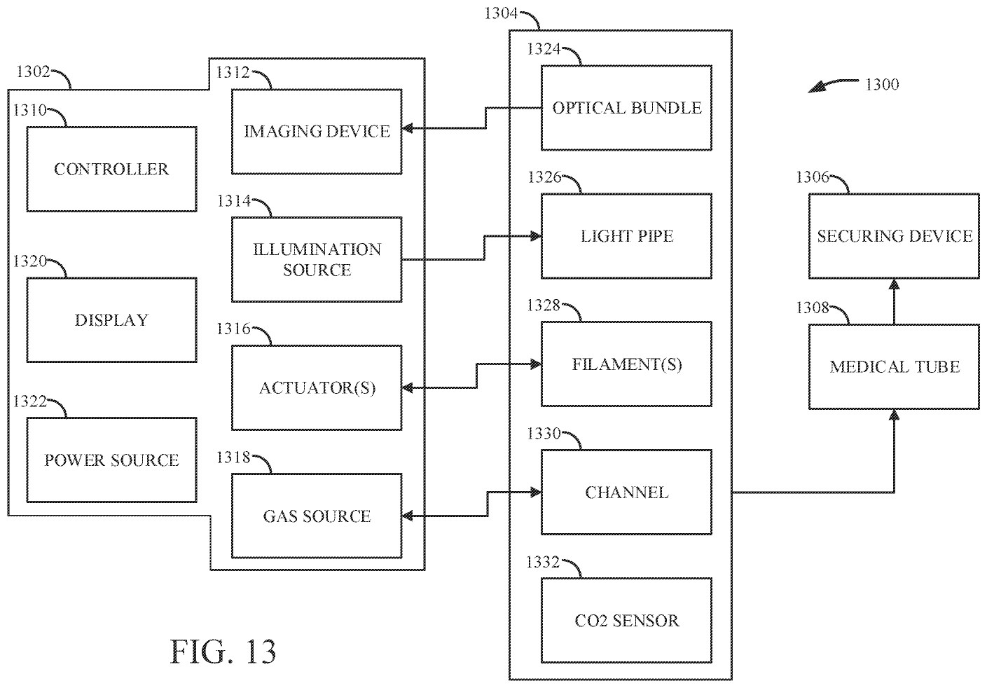

FIG. 13 shows a schematic illustration of a block diagram of a guidance system 1300. The guidance system 1300 can include a robotic system 1302, a stylet 1304 (or in other words a guidewire), a securing device 1306, and a medical tube 1308. The robotic system 1302 can be implemented in a similar manner as the device 400 (e.g., the robotic control and display center), or other robotic systems described herein. In some embodiments, the robotic system 1302 can include a controller 1310, an imaging device 1312, an illumination source 1314, one or more actuators 1316, a gas source 1318, a display 1320, and a power source 1322. In some cases, the controller 1310 can be implemented in a similar manner as the controller 406, the imaging device 1312 can be implemented in a similar manner as the imaging devices 108, 208, and the illumination source 1314 can be implemented in a similar manner as the light source 220. For example, the controller 1310 can include one or more processors, the imaging device 1312 can include an imaging sensor (e.g., a CCD, and active pixel sensor, etc.), and the illumination source 1314 can include one or more light sources (e.g., one or more LEDs).

In some configurations, the robot system 1302 can include five actuators 1316, however, in other configurations, the robot system 1302 can include other numbers of the actuators 1316 (e.g., one, two, three, four, etc.). The actuators 1316 can be implemented in different ways. For example, an actuator 1316 can include a motor (e.g., an electric motor), and an extender (e.g., a lead screw) coupled to the motor, in which rotation of the motor drives extension (and retraction) of the extender. Thus, in some cases each actuator 1316 can be a linear actuator. As another example, each actuator 1316 can be implemented as just a motor in which rotation of the motor drives rotation of component coupled to the motor. As described in more detail below, the one or more actuators 1316 are configured to adjust the extension and orientation of the stylet 1304 (e.g., as the stylet 1304 advances into the patient). In some embodiments, each actuator 1316 can include a stepper motor.

The gas source 1318 can be implemented in different ways. For example, the gas source 1318 can be configured to drive fluid (e.g., oxygen, carbon dioxide, etc.) into the stylet 1304, to drive fluid out of the stylet 1304, or both. For example, the gas source 1318 can be an oxygen source (e.g., a pressurized oxygen source) and the gas source 1318 can introduce oxygen into the stylet 1304. In this case, the gas source 1318 can include a valve (e.g., a solenoid valve) controllable by the controller 1310 to selectively open (or close) the valve (and to varying degrees) to adjust the flow rate of oxygen into the stylet 1304. As another example, the gas source 1318 can include a pump that is configured to draw fluid out of the patient, through the stylet 1304, and into a reservoir (e.g., of the robot system 1302). In some cases, the gas source 1318 can include the oxygen source and the pump (e.g., a suctioning pump) and can switch between them (e.g., via the controller 1310) to selectively cause oxygen delivery to the stylet 1304, or to cause fluid to flow from the patient through the stylet 1304, and back to the robot system 1302 (e.g., to the reservoir). For example, the gas source 1318 can include one or more valves each of which can be controllable by the controller 1310 between a first configuration and a second configuration. In the first configuration, the controller 1310 can cause the one or more valves to cause oxygen to flow from the oxygen source into the stylet 1304 (and into the patient) and to block fluid from flowing from the patient, through the stylet 1304, and into the robot system 1302. In the second configuration, the controller 1310 can cause the one or more valves to block oxygen from flowing from the oxygen source and into the stylet 1304, and to allow fluid to flow from the patient, through the stylet 1304, and into the robot system 1302.

In some cases, the display 1320 and the power source 1322 can be implemented in a similar manner as the display 414 and the power source 102, respectively. For example, the display 1320 can be an LCD display, a touchscreen display, an LED display, an OLED display, etc. As another example, the power source 1322 can be an electrical power source, including for example, a battery (e.g., a lithium-ion battery). In some configurations, the power source 1322 can provide power to each component of the robotic system 1302, as appropriate. For example, the power source 1322 can provide power to the controller 1310, the imaging device 1312, the illumination source 1314, the actuators 1316, the gas source 1318, and the display 1320. In some configurations, although not shown in FIG. 13, the robot system 1302 can be configured to communicate (e.g., bi-directionally communicate) with other computing devices (not shown). For example, the robot system 1302 can communicate with a computer to, for example, transmit data (e.g., imaging data) to the computer for subsequent analysis. In this way, the robot system 1302 can utilize other computational resources for faster analysis. In some cases, then, the robot system 1302 can include a communication system (e.g., a wireless module, a serial module, etc.), including for example, an antenna, a receiver, a transceiver, etc., to facilitate communication to other computing devices.

In some embodiments, although not shown in FIG. 13, the robot system 1302 can include a spectrometer (e.g., the spectrometer 104), other processing circuitry including a GPU (e.g., the GPU 404), etc. In some configurations, each of the components of the robot system 1302 can be integrated within a single housing of the robot system 1302, however some components of the robot system 1302 can be external to the single housing. For example, the display 1320 can be external to the housing of the robot system 1302 (e.g., as part of a separate computing device), the gas source 1318 can be enteral to the housing of the robot system 1302, etc. Although FIG. 13 illustrates the robot system 1302 has having a single imaging device 1312, a single illumination source 1314, a single gas source 1318, the robot system 1302 can include other numbers of these components. For example, the robot system 1302 can include multiple imaging devices 1312, multiple illumination sources 1314, multiple gas sources 1318 (e.g., an oxygen gas source and a CO2 gas source). Similarly, the robot system 1302 can include multiple spectrometers.

The stylet 1304 can be implemented in a similar manner as the previously described stylets including, for example, the articulated stylets 420, 502, 702, 804, 900, 1001, 1118, and thus these stylets pertain to the stylet 1304 (and vice versa). The stylet 1304 can include an optical bundle 1324, a light pipe 1326 (or in other words a light guide), one or more filaments 1328, a channel 1330, and a carbon dioxide (“CO2”) sensor 1332. The optical bundle 1324 can include one or more optical fibers (e.g., coherent optical fibers), each of which is configured to direct light from the interior of the patient and to one or more optical sensors of the robot system 1302. For example, a first optical fiber of the optical bundle 1324 can be optically coupled to the imaging device 1312 of the robot system 1302 (e.g., when the stylet 1304 is coupled to the robot system 1302 (e.g., at the housing of the robot system 1302). Similarly, a second optical fiber of the optical bundle 1324 can be optically coupled to the spectrometer of the robot system 1302. In these ways, light from the inside of the patient can be imaged by the imaging device 1312 without the stylet 1304 requiring to include an imaging device (or spectrometer). Thus, advantageously, the stylet 1304 can be disposed of after the procedure and the robot system 1302 can be reused for a subsequent procedures (e.g., because the imaging device 1312 or spectrometer have not come in direct contact with the patient during the procedure). As such, because the stylet 1304 does not include the imaging device 1312, the spectrometer, other expensive components, etc., the stylet 1304 can be made considerably more cost-effective (e.g., cheaper) while still, via the robot system 1302, being able to acquire images of the interior of the patient to guide advancement of the stylet 1304.

Similarly to the optical bundle 1324, the light pipe 1326 can also be optically coupled to the illumination source 1314 of the robot system 1302. In this way, light emitted by the illumination source 1314 can be emitted into the light pipe 1326, travel through the light pipe 1326, and can be emitted out of the light pipe 1326 and into the interior of the patient. In some cases, including when the stylet 1304 includes multiple light pipes 1326, each light pipe 1326 can be optically coupled to a corresponding illumination source 1314 of the robot system 1302 (e.g., when the robot system 1302 include multiple illumination sources 1314). In some configurations, while the light pipe 1326 can be advantageous for similar reasons as the optical bundle (e.g., the illumination source 1314 can be reused), in other configurations, the stylet 1304 can include the one or more illumination sources 1314 (rather than the robot system 1032). In this case, for example, each illumination source 1314 can be coupled to a distal end of the stylet 1304, and can be electrically connected to the robot system 1302. For example, electrical wires can be routed from the illumination source 1314 to the power source 1322 and to the controller 1310. Regardless of the configuration, the controller 1310 can selectively cause each illumination source 1314 to emit light thereby illuminating (or ceasing to illuminate) the interior of the patient.

In some configurations, the stylet 1304 can include one or more lenses. For example, a first lens (e.g., a converging lens) can be optically coupled to one optical fiber of the optical bundle 1324. In particular, the first lens can be positioned in front of distal end of the one optical fiber of the optical bundle 1324. In this way, light from the interior of the patient can be focused into the distal end of the one optical fiber, which can advantageously increase the field of view of the imaging device 1312. In some cases, the first lens can be coupled to the stylet 1304 (e.g., at a distal end of the stylet 1304). In some configurations, the first lens can be positioned within the stylet 1304. As another example, a second lens (e.g., a converging lens) can be optically coupled to a second optical fiber of the optical bundle 1324, and can be positioned in front of the distal end of the second optical fiber. In this way, light from inside the patient can be focused into the distal end of the second optical fiber, which can increase the amount of light received by the spectrometer of the robot system 1302. As yet another example, a third lens (e.g., a diverging lens) can be optically coupled to a distal end of the light pipe 1326 (or a distal end of the illumination source 1314). In this way, light emitted from the light pipe 1326 (or the illumination source 1314) can be dispersed by the third lens to better illuminate the interior of the patient, which can facilitate better image acquisition by the imaging device 1312.

In some embodiments, the stylet 1304 can include the one or more filaments 1328. For example, the stylet 1304 can include four filaments 1328 or other numbers of filaments including, for example, one, two, three, five, etc. Each filament 1328 can be coupled to, or integrated within, a portion of the body of the stylet 1304 (or the entire longitudinal extent of the body of the stylet 1304). For example, a portion of each filament 1328 can be coupled to (or integrated within) the body of the stylet 1304 at a distal end of the body of the stylet 1304. The remaining extent of each filament 1328 can then be decoupled from the body of the stylet 1304, so that when each filament 1328 is tensilely loaded (e.g., pulled in tension), the distal end of the stylet 1304 in which each filament 1328 is coupled to, deflects towards the filament pulled in tension (e.g., with the amount of deflection corresponding to the amount of tension). In some cases, an end of each filament 1328 opposite the end coupled to the body of the stylet 1304 can be coupled to a respective actuator 1316. In this way, the controller 1310 can cause the actuator 1316 to pull a respective filament 1328 in tension to adjust the deflection orientation of a distal end of the stylet 1304.

In some embodiments, the filaments 1328 can be positioned relative to the body of the stylet 1304 in different ways. For example, the body of the stylet 1304 can extend along a longitudinal axis, and a first filament 1328 can be positioned at substantially (i.e., deviating by less than 10 percent) 0 degrees around the longitudinal axis, a second filament 1328 can be positioned at substantially 90 degrees around the longitudinal axis, a third filament 1328 can be positioned at substantially 180 degrees around the longitudinal axis, and a fourth filament 1328 can be positioned at substantially 270 degrees around the longitudinal axis. In this configuration, the first, second, third, and fourth filaments 1328 can form a square (or rectangle) shape in cross-section (e.g., in an axial cross section taken at a portion of the longitudinal axis). While this is one configuration of positioning of the filaments 1328 others are possible. For example, the filaments 1328 can collectively form other shapes in an axial cross-section including a triangle, a hexagon, etc. In some embodiments, the filaments 1328 can be implemented in different ways. For example, each filament 1328 can be a single thread (e.g., of a polymer), a braided thread, a wire, a braided wire, etc.

In some embodiments, the stylet 1304 can include the channel 1330, which can extend entirely through the stylet 1304 (e.g., from a proximal end of the stylet 1304 to a distal end of the stylet 1304). For example, the channel 1330 can extend through the body of the stylet 1304 from one end to an opposing end of the body. In some cases, the channel 1330 can house some of the components of the stylet 1304. For example, the optical bundle 1324 can be positioned within the channel 1330, the light pipe 1326 can be positioned within the channel 1330, and the CO2 sensor can be positioned within the channel 1330. In some cases, the channel 1330 can be in fluid communication with the gas source 1318 (or gas sources 1318). In this way, fluid (e.g., oxygen, CO2, etc.) from the gas source 1318 can flow into and through the channel 1330 into the interior of the patient, or fluid can flow from the interior of the patient, into and through the channel 1330 and back to the robot system 1302 (or other reservoir), such as when the gas source is suction source 1318 is a suction source.

In some embodiments, the stylet 1304 can include the CO2 sensor 1332. The CO2 sensor 1332 can be implemented in different ways. For example, the CO2 sensor 1332 can be a nondispersive infrared sensor, a photoacoustic spectroscopy sensor, a chemical CO2 sensor, etc., each of which is configured to sense an amount of CO2 in fluid communication with the CO2 sensor 1332. The CO2 sensor 1332 can be coupled to the body of the stylet 1304 (e.g., at a distal end of the stylet 1304). In this way, as the stylet 1304 is advanced into the patient, the CO2 sensor is in fluid communication with the interior volume of the patient. In some cases, the position of the CO2 sensor 1332 on the stylet 1304 can substantially correspond to the position of the distal end of the optical bundle on the stylet 1304. In this way, the images acquired by the imaging device 1312 can correspond to a substantially similar location as the CO2 amount sensed by the CO2 sensor 1332. In some cases, the CO2 sensor 1332 can be electrically connected (e.g., via wires) to the power source 1322 and to the controller 1310 of the robot system 1302. In this way, the controller 1310 can receive CO2 amounts from the CO2 sensor 1332 and adjust the extension or orientation of the distal end of the stylet 1304 based on the CO2 amount.

In some configurations, the stylet 1304 can have a distal end and a proximal end. The proximal end of the stylet 1304 can be removably coupled to the housing of the robot system 1302. When the proximal end of the stylet 1304 is coupled to the housing of the robot system 1302, each optical fiber optically couples to a respective imaging device (or spectrometer), the light pipe 1326 optically couples to the illumination source 1314, the channel 1330 fluidly communicates with the gas source 1318, and the CO2 sensor electrically connects to the controller 1310. In some cases, when the proximal end of the stylet 1304 is coupled to the housing of the robot system 1302, each filament 1328 couples to a respective actuator 1316 (e.g., an extender of the respective actuator). For example, an end of each filament 1328 can have a clip that engages with a corresponding clip of an extender of a respective actuator 1316 to couple the each filament 1328 to the respective actuator 1316 (e.g., when the proximal end of the stylet 1304 is coupled to the housing of the robotic system 1302). In these ways, the stylet 1304 can be easily interfaced with the robot system 1302 for one procedure, can be removed (for disposal), and a subsequent stylet (e.g., similar to the stylet 1304) can be easily interfaced with the robot system 1302 for a subsequent procedure (e.g., on a different patient). In some embodiments, the stylet 1304 can be coiled, and an actuator 1316 implemented as a motor can engage and thus rotate the stylet 1304 in the coiled configuration. In this way, by rotating the motor, the coiled stylet 1304 rotates (e.g., unraveling the coil), and the distal end of the stylet advanced away from the proximal end of the coiled stylet 1304.

In some embodiments, the stylet 1304 and the components of the stylet 1304 can be configured to be articulated. For example, the body of the stylet 1304, the optical bundle 1324, the light pipe 1326, etc., can each be configured to be articulated to different orientations. For example, a distal end of the stylet 1304, including the optical bundle 1324, the light pipe 1326, can be configured to curve to adjust the orientation of the distal end of the stylet 1304.

In some configurations, the guidance system 1300 can include the securing device 1306 and the medical tube 1308. The securing device 1306 can be configured to selectively prevent (and allow) movement of the medical tube 1308 relative to the securing device 1306. In addition, the securing device 1306 can be removably coupled to the patient. In some cases, the securing device 1306 can include a lock that can move between a first position and a second position. With the lock in the first position, the medical tube 1308 (or other device) is allowed to advance further into the securing device 1306 and thus further into the interior of the patient. However, with the lock in the second position, the medical tube 1308 is blocked from advancing further into the securing device 1306 and thus blocked from advancing further into the interior of the patient. In other words, the lock in the first position allows relative movement between the securing device 1306 and the medical tube 1308, while the lock in the second position block relative movement between the securing device 1306 and the medical tube 1308.

In some embodiments, the guidance system 1300 can include an oropharyngeal device described herein. In this case, the oropharyngeal device can be inserted into the patients mouth, as described below, and the securing device 1306 can subsequently be secured to the patient (and coupled to the securing device 1306).

As shown in FIG. 13, the distal end of the stylet 1304 can be inserted into the medical tube 1308 and advanced into the securing device 1306. Then, the stylet 1304 can be advanced into the patient and can be guided by the robot system 1302 until the distal end of the stylet 1304 reaches the desired location. At that point, the medical tube 1308 can be advanced along the stylet 1304 until the medical tube 1308 reaches the distal end of the stylet 1304. In some cases, the stylet 1304 can include a mechanical stop (e.g., a protrusion, recess, etc.) positioned at the distal end of the stylet 1304, which can block further advancement of the medical tube 1308 past the mechanical stop. After the medical tube 1308 has been placed, the stylet 1304 can be removed (e.g., using actuator commands that were opposite to driving the stylet 1304 to the desired location).

FIG. 14 shows a schematic illustration of a side view of a stylet 1400, which can be a specific implementation of the stylet 1304 (or other stylets described herein). The stylet 1400 can include a body 1402 defining a distal end 1404 and a proximal end 1406, an optical bundle 1408, a light pipe 1410 filaments 1412, 1414, 1416, 1418, a channel 1420, and a CO2 sensor 1422 As shown in FIG. 14, the body 1402 extends along the longitudinal axis 1424.

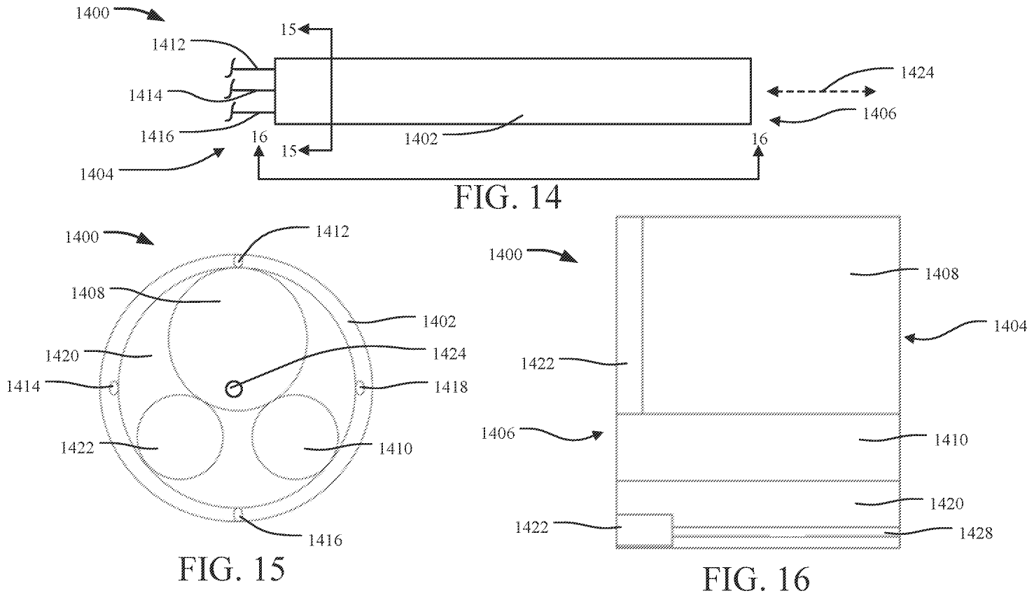

FIG. 15 shows an axial cross-sectional view of the stylet 1400 taken along line 15-15 of FIG. 14. As shown in FIG. 15, each filament 1412, 1414, 1416, 1418 is embedded into the body 1402 of the stylet 1400, however, in other configurations, each filament 1412, 1414, 1416, 1418 can be coupled to the body 1402. The filaments 1412, 1414, 1416, 1418 are positioned around the longitudinal axis 1424 so that the filaments 1412, 1414, 1416, 1418 surround the longitudinal axis 1424. For example, the filament 1412 is positioned at substantially 0 degrees around the longitudinal axis 1424, the filament 1414 is positioned at substantially 90 degrees around the longitudinal axis 1424, the filament 1416 is positioned at substantially 180 degrees around the longitudinal axis 1424, and the filament 1418 is positioned at substantially 270 degrees around the longitudinal axis 1424. Each filament 1412, 1414, 1416, 1418 is configured to be coupled to a corresponding actuator and as a respective actuator pulls a corresponding filament, the filament causes the stylet 1400 to deflect towards the pulled filament. In this way, with the filaments 1412, 1414, 1416, 1418 positioned accordingly relative to the body 1402, the stylet 1400 can be oriented in substantially any direction (e.g., without having to rotate the stylet 1400 around the axis 1424). In some cases, including when there are three filaments 1412, 1414, 1416 arranged in a triangle (e.g., with one filament positioned at 0 degrees around the axis 1424, with another filament positioned at 120 degrees around the axis 1424, and with another filament positioned at 240 degrees around the axis 1424), the stylet 1400 can be oriented in substantially any direction (e.g., without having to rotate the stylet 1400 around the axis 1424).

As shown in FIG. 15, the optical bundle 1408 is positioned within the channel 1420 that extends through the body 1402, the light pipe 1410 (or an illumination source, including the illumination source 1314) is positioned within the channel 1420, and the CO2 sensor 1422 is positioned within the channel 1420. In some cases, the optical bundle 1408, the light pipe 1410, and the CO2 sensor 1422 can be coupled to the body 1402 (e.g., an inner of the body 1402). In some embodiments, the body 1402 can have an outer width of less than or equal to 4.45 millimeters, and the length of the body 1402 can be less than or equal to 953 mm.

FIG. 16 shows a longitudinal cross-section of the stylet 1400 taken along line 16-16 of FIG. 14. As shown in FIG. 16, a distal end of the optical bundle 1408 is optically coupled to one or more lenses each of which is positioned proximal to the distal end of the stylet 1400. For example, a first lens (e.g., a focusing lens) can be positioned at the distal end of the stylet 1400 in front of a distal end of the optical bundle 1408, and can be optically coupled to an optical fiber of the optical bundle 1408, which can also be optically coupled to an imaging device (e.g., the imaging device 1312). In this way, light can be focused by the first lens into the optical fiber. Regardless of the configuration, the distal end of the optical bundle 1408 can be positioned proximal to the distal end of the stylet 1400 so that light within the patient can be emitted into the distal end of the optical bundle, travel though the optical bundle to be received by the imaging device (e.g., to acquire imaging data).

As shown in FIG. 16, the CO2 sensor 1422 is positioned proximal to the distal end of the stylet 1400. In this way, the CO2 sensor 1422 can be in fluid communication with the interior of the patient to sense the CO2 amount at the location of the CO2 sensor 1422 (e.g., inside the patient). As shown in FIG. 16, the CO2 sensor 1422 can be electrically connected to one or more wires 1428, which can be electrically connected to a robot system (e.g., the robot system 1302, and in particular, the controller 1310).

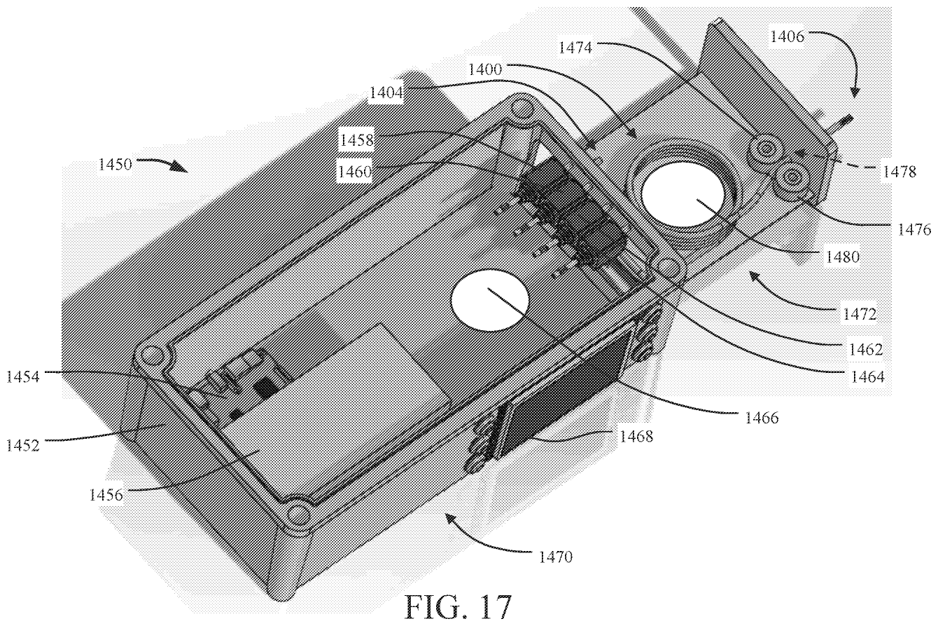

FIG. 17 shows a top isometric view of a robot system 1450 in an open configuration, which can be a specific implementation of the robot system 1302 (or others) described herein. The robot system 1450 can include a housing 1452, a controller 1454, a power source 1456, actuators 1458, 1460, 1462, 1464, 1466, a display 1468. The housing 1452 can have a main body 1470, and a cartridge 1472 that is removably coupled to the main body 1470. As shown in FIG. 17, the cartridge 1472 can support the stylet 1400. For example, the cartridge 1472 can include a protrusion 1480 in which the stylet 1400 is coiled around. In some cases, the cartridge 1472 can include rollers 1474, 1476, each of which can contact and guide the stylet 1400 to advance through the hole 1478 in the cartridge 1472. In some configurations, the display 1468 can be coupled to the main body 1470 of the housing 1452.

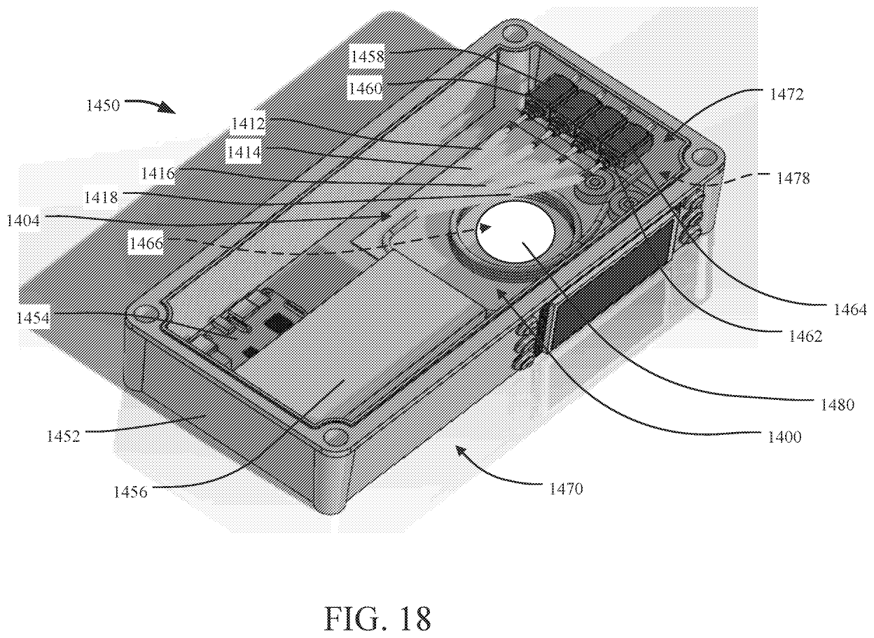

FIG. 18 shows a top isometric view of the robot system 1450 in a closed configuration. As shown in FIG. 18, the cartridge 1472 has been slid into the interior volume of the main body 1470. In some cases, the cartridge 1472 can be locked when the cartridge 1472 is slid into the main body 1470 (e.g., when the cartridge 1472 includes a clip). When the cartridge 1472 is engaged with the main body 1470 (e.g., the housing 1452 being in a closed configuration), the protrusion 1480 aligns with an engages the motor of the actuator 1466 (e.g., the motor contacting the protrusion 1480). In this way, the controller 1454 can cause the motor 1466 to rotate, thereby rotating the protrusion 1480, and thus advancing the distal end 1406 of the stylet 1400 through the hole 1478 and away from the housing 1452.

As shown in FIG. 18, when the cartridge 1472 is engaged with the main body 1470 so that the housing 1452 is in the closed configuration, each filament 1412, 1414, 1416, 1418 can be coupled to an extender of a respective actuator 1458, 1460, 1462, 1464. For example, a proximal end of the filament 1412 can be coupled to the extender of the actuator 1458, a proximal end of the filament 1414 can be coupled to the extender of the actuator 1460, a proximal end of the filament 1416 can be coupled to the extender of the actuator 1462, and a proximal end of the filament 1418 can be coupled to the extender of the actuator 1464. In operation, the controller 1454 can cause each actuator 1458, 1460, 1462, 1464 to extend or retract thereby adjusting the tensile force on the respective filament 1412, 1414, 1416, 1418, thereby adjusting the orientation of the stylet 1400 relative to the housing 1452 (e.g., with greater tensile forces on a filament urging the opposing end of the filament and thus the body of the stylet coupled to the filament closer to the filament). In some cases, the proximal end 1404 of the stylet 1400 can be positioned within the housing 1452, while the distal end 1406 can be positioned outside the housing 1452.

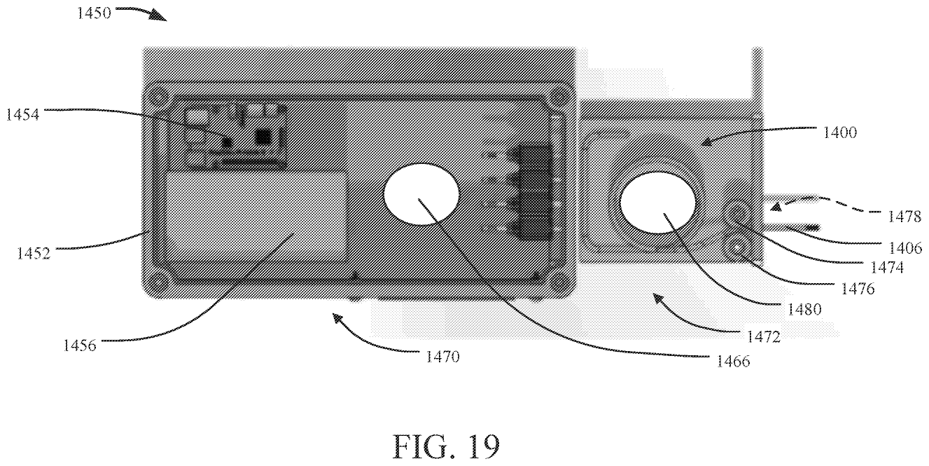

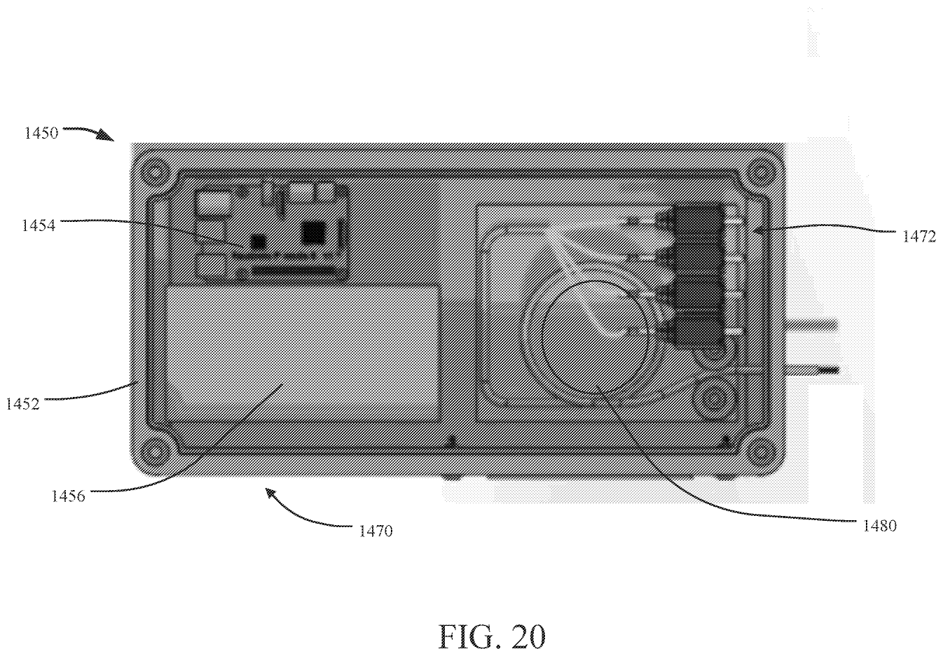

FIGS. 19 and 20 show alternative views of the robot system 1450. For example, FIG. 19 shows a top view of the robot system 1450 in an open configuration, while FIG. 20 shows a top view of the robot system 1450 in a closed configuration. As shown in FIG. 19, the cartridge 1472 is positioned outside of the internal volume of the main body 1470, with the stylet 1400 being pre-coiled around the protrusion 1480 and with the distal end 1406 of the stylet 1400 extending through the hole 1478 (and being in contact with the rollers 1474, 1476). As shown in FIG. 20, the cartridge 1472 has slid into the internal volume of the main body 1470 (e.g., by pushing the cartridge 1472 towards the main body 1470, when the robot system 1450 is in the open configuration). Although not shown in FIGS. 17-20, the robot system 1450 can also include an imaging device, a spectrometer, an illumination source, and a gas source. In some cases, when the robot system 1450 is in a closed configuration (e.g., the cartridge 1472 being inserted into the main body 1470, each optical fiber of the optical bundle 1408 can optically couple to a respective optical component of the robot system 1450 (e.g., an imaging device, or a spectrometer), the light pipe 1410 can optically couple to the illumination source of the robot system 1450 (or the illumination source of the stylet can electrically connect to the robot system 1450 including the controller 1454), and the CO2 sensor 1422 can electrically connect to the robot system 1450 (e.g., the controller 1454).

In some embodiments, including when the procedure has been completed and the stylet 1450 has been placed (and the patient intubated), the cartridge 1472 can be disposed, and an additional cartridge 1472 can be loaded into engagement with the main body 1470. In this way, more expensive components of the robot system 1450, including an imaging device, a spectrometer, illumination sources, power sources, gas sources, actuators, etc., do not have to be disposed of after use. Rather, less expensive components of the stylet 1400 including an optical bundle, a light pipe, filaments, a CO2 sensor, etc., can be disposed of after the procedure is completed. In this way, contamination concerns are avoided as the stylet 1400 can simply be discarded, rather than requiring cleaning.

While the robot system 1450 has been described with the cartridge 1472 being removably coupled to (and from) the main body 1470, in other cases, the cartridge 1472 can be coupled to the main body 1470 so that the housing 1452 is a monolithic between the main body 1470 and the cartridge 1472. In this configuration, for example, a stylet 1400 can be loaded into the housing 1452, optically coupled with the desired components, and coupled to the desired components (e.g., coupling each filament to each actuator). Then, after the procedure has been completed, the stylet 1400 can be disposed of, and an additional stylet can be engaged according to the procedure above for a subsequent procedure (e.g., for a different patient).

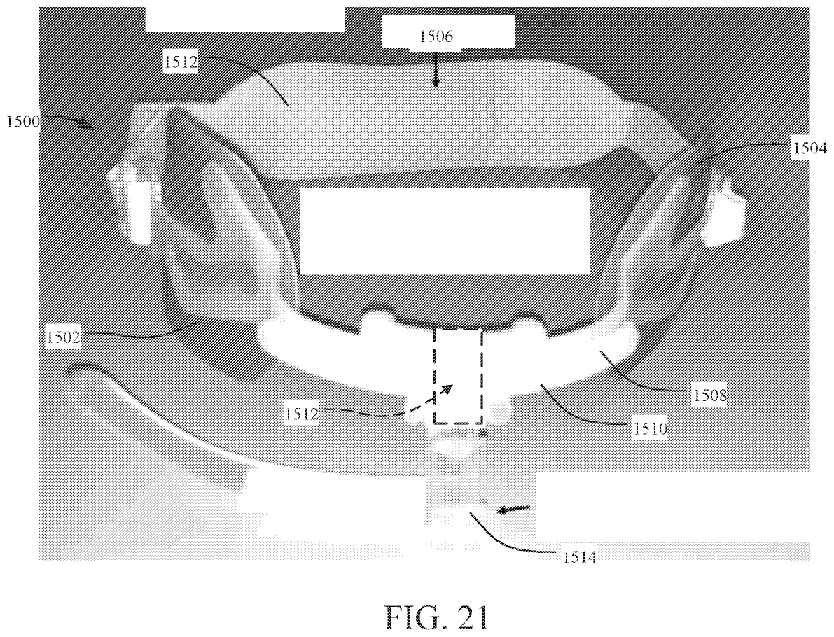

FIG. 21 shows a front isometric view of a securing device 1500, which can be a specific implementation of the securing device 1306. For example, the securing device 1500 can be an endotracheal tube securing device (e.g. configured to secure an endotracheal tube to the securing device 1500). The securing device 1500 can include pads 1502, 1504, 1506, one or more ties 1508, a housing 1510 having a channel 1512 therethrough, and a bracket 1514. The housing 1510 can be coupled to the pads 1502, 1504. For example, a first end of the housing 1510 can be coupled to the pad 1502, and a second end of the housing 1510 can be coupled to the pad 1504. The pad 1506 can also be coupled to the pads 1502, 1504. For example, a first end of the pad 1506 can be inserted into a slot of the pad 1502 and can be coupled to a different portion of the pad 1506, while a second end of the pad 1506 can be inserted into a slot of the pad 1504 and can be coupled to a different portion of the pad 1506. The tie 1508 can be coupled to the housing 1510 (e.g., at opposing ends of the housing 1510), and in some cases, a first end of the tie 1508 can be coupled to the pad 1502, and a second end of the tie 1508 can be coupled to the pad 1504. In some cases, the tie 1508 can be structured as a zip-tie. Regardless of the configuration, the tie 1508 (and ends of the pad 1506) can be pulled (or otherwise tied) to decrease the periphery (e.g., circumference) of the securing device 1500 to secure the securing device 1500 to the patient. In some cases, each of the pads 1502, 1504 can include an adhesive layer so that each of the pads 1502, 1504 can be removably coupled to the patient. For example, the adhesive of the pad 1502 can couple the pad 1502 to a first cheek of the patient, and the adhesive of the pad 1504 can couplet the pad 1504 to the second cheek of the patient. Then, with the pads 1502, 1504 secured, the ends of the pad 1506 and the tie 1508 can be tightened to secure the securing device 1500 to the patient. As shown in FIG. 21, the channel 1512 extends through the housing 1510 and can be configured to receive an endotracheal tube. In some cases, the bracket 1514 can be coupled to the housing 1510 and can be positioned above the channel 1512 to provide a securement location for the endotracheal tube. For example, after the endotracheal tube has been inserted into the channel 1512, the endotracheal tube can be coupled (e.g., via a tie) to the bracket 1514 to block relative movement between the endotracheal tube and the securing device 1500.

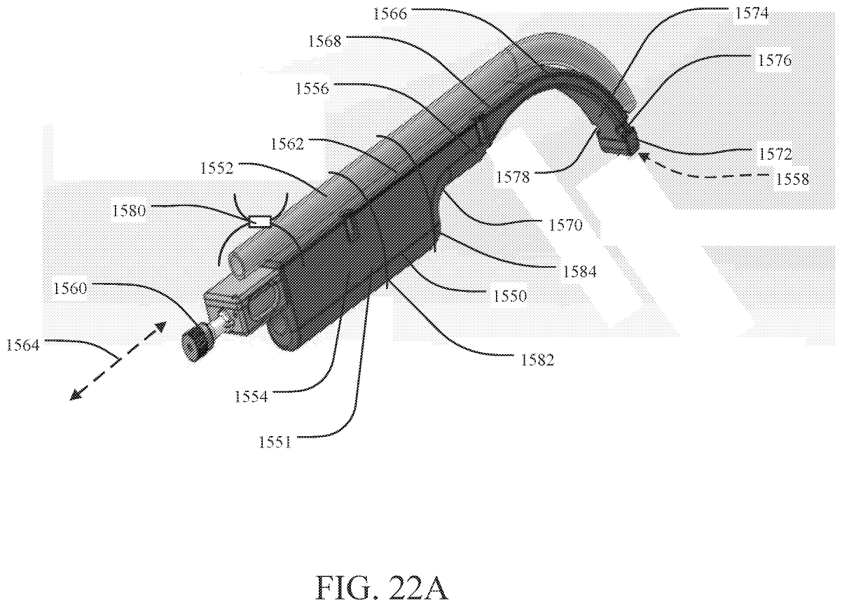

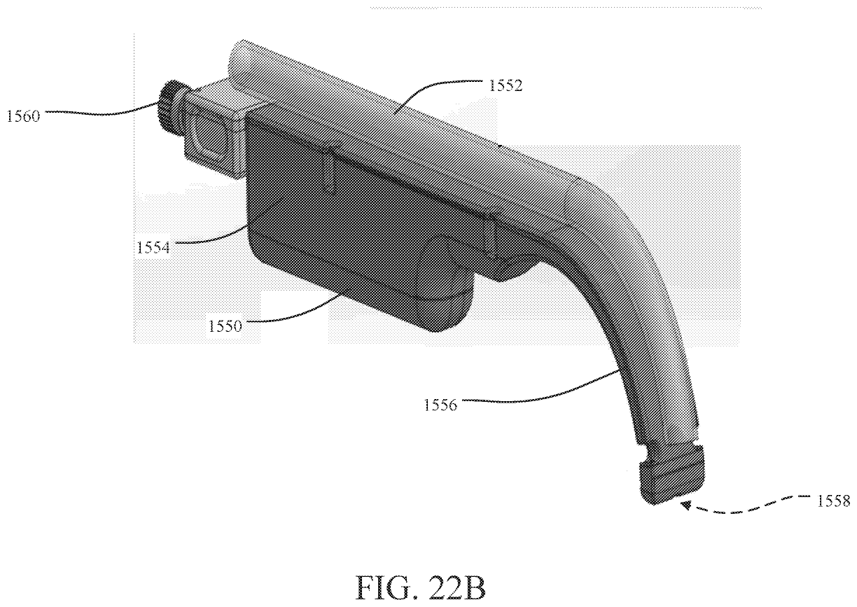

FIGS. 22A and 22B show isometric views of an oropharyngeal device 1550 with an endotracheal tube 1552 engaged with the oropharyngeal device 1550. In particular, FIG. 22A shows a front isometric view of the oropharyngeal device 1550, while FIG. 22B shows a rear isometric view of the oropharyngeal device 1550. The oropharyngeal device 1550 can include a body 1551 having a handle 1554 and a mouthpiece 1556, a conduit 1558 that extends through the body 1551, and a port connector 1560 in fluid communication with the conduit 1558. The handle 1554 can have a greater cross-sectional height than the mouthpiece 1556. In this way, when the oropharyngeal device 1550 is placed into the mouth of the patient, the handle 1554 is blocked from moving into the mouth of the patient. In some cases, the handle 1554 can have an upper surface 1562 that is substantially straight along the length of the handle 1554.

As shown in FIG. 22A, the mouthpiece 1556, which is coupled to the handle 1554, is curved along a longitudinal axis 1564 in which the handle 1554 extends along to define a curved section 1566 of the mouthpiece 1556. In other words, a distal end 1572 of the mouthpiece curves away from the longitudinal axis 1564 with the handle 1554 and the mouthpiece 1556 extending within the same longitudinal plane (e.g., that includes the longitudinal axis 1564). A substantially straight section 1568 of the mouthpiece 1556 (e.g., extending substantially straight along the longitudinal axis 1564) is coupled to (or integrally formed with) the distal end of the handle 1554 at a first end of the section 1568, while the section 1568 at an opposite second end is coupled (or integrally formed with) the proximal end of the curved section 1566. In some configurations, the mouthpiece 1556 can include a notch 1570 (e.g., at the mouthpiece 1556 proximal to the distal end of the handle 1554), which defines a decrease in cross-sectional height for the mouthpiece 1556. Similarly, the curved section 1566 of the mouthpiece has a smaller cross-sectional height than the cross-sectional height of the handle 1554 (and the cross-sectional height of the section 1568 of the handle 1554). In some cases, the cross-sectional height of the oropharyngeal device 1550 decreases along the longitudinal axis 1564 in a direction from the proximal end of the oropharyngeal device 1550 to a distal end of the oropharyngeal device 1550 (e.g., that includes the distal end 1572 of the mouthpiece 1556).

In some embodiments, the conduit 1558 extends entirely through the oropharyngeal device 1550, from the proximal end of the oropharyngeal device 1550 to the distal end of the oropharyngeal device 1550. For example, the conduit 1558 can extend through the handle 1554, through the mouthpiece 1556, and out the mouthpiece 1556. The conduit 1558 is in fluid communication with the port connector 1560, and the port connector 1560 is configured to be interface with an oxygen gas source (e.g., one of the gas sources 1318). In this way, oxygen gas (from the oxygen gas source) is configured to flow into the port connector 1560, through the conduit 1558, and out the conduit 1558 into the trachea of the patient. Thus, oxygen gas can be delivered to the patient's airway even while placing the endotracheal tube 1552.

In some embodiments, the curved section 1566 of the mouthpiece can contour the curvature of the oropharyngeal cavity. For example, when the oropharyngeal device 1550 is placed into engagement with the patient, the curved section 1566 curves along the structures that define the oropharyngeal cavity. In some configurations, a lower surface 1574 of the curved section 1566 of the mouthpiece 1556 is substantially flat. In this way, when the oropharyngeal device 1550 is placed, a greater surface area of the lower surface 1574 (e.g., as opposed to the lower surface 1574 being rounded) contacts the structures that define the oropharyngeal cavity of the patient to prevent relative movement between the oropharyngeal device 1550 and the patient. In some configurations, the distal end 1572 of the curved section 1566 can include notches 1576, 1578. The notches 1576, 1578 are directed into opposing sides of the curved section 1566. For example, the notch 1576 is directed into a first lateral side of the curved section 1566 towards the longitudinal axis 1564, while the notch 1578 is directed into a second lateral side of the curved section 1566 towards the longitudinal axis 1564. In some cases, the notches 1576, 1578 can provide securing locations for a tie, to couple the endotracheal tube 1552 to the oropharyngeal device 1550.

In some embodiments, the oropharyngeal device 1550 can include a channel that extends along the longitudinal axis 1564. For example, the channel can be directed into the upper surface 1562 of the handle 1554, an upper surface of the section 1568 of the mouthpiece 1556, and an upper surface of the curved section 1566. In some cases, the channel can extend partially (or entirely) along the handle 1554, along the section 1568 of the mouthpiece 1556, and along the curved section 1566 of the mouthpiece 1556, in the longitudinal direction. Regardless of the configuration, the channel can be configured to receive the intubation tube 1552. In this way, the channel can cradle the intubation tube 1552 (e.g., preventing relative movement between the components) and can guide placement of the endotracheal tube 1552. For example, after the oropharyngeal device 1550 is placed, the endotracheal tube 1552 can be placed into the channel and advanced towards the distal end 1572, with the channel guiding advancement of the intubation tube 1552.

In some embodiments, the endotracheal tube 1552 can be secured to the oropharyngeal device 1550. For example, the oropharyngeal device 1550 can include a clip 1580, and ties 1582, 1584. The clip 1580 can include or more arms that retractably engage the endotracheal tube 1552 and the oropharyngeal device 1550 to secure the endotracheal tube 1552 to the oropharyngeal device 1550. In some cases, the clip 1580 can couple the endotracheal tube 1552 to the oropharyngeal device 1550 at the handle 1554 of the oropharyngeal device 1550. The ties 1582, 1584 can also secure the endotracheal tube 1552 to the oropharyngeal device 1550. For example, each tie 1582, 1584 can wrap around the endotracheal tube 1552 and the oropharyngeal device 1550 (e.g., at the handle 1554) to secure the endotracheal tube 1552 to the oropharyngeal device 1550. As shown in FIG. 22A, the ties 1582, 1584 and the clip 1580 are separated from each other along the longitudinal axis 1564. In some embodiments, the oropharyngeal device 1550 can include multiple clips (e.g., multiple clips 1580) each separated from each other along the longitudinal axis 1564. Similarly, the oropharyngeal device 1550 can include additional ties (e.g., ties similar to the ties 1582, 1584), including one, two, three, four, etc., each of which is separated along the longitudinal axis.

In some embodiments, the oropharyngeal device 1550 can be placed into the mouth of the patient and can retract the tongue and can contact the structures that define the larynx to expose the larynx (e.g., increasing the cross-section of the laryngeal cavity). For example, a practitioner can grab the handle 1554 and can insert the distal end of the curved section 1566 into the mouth of the patient, and then into the throat of the patient. When the oropharyngeal device 1550 is placed, the mouthpiece 1556 is positioned within the mouth of the patient with the lip of the patient contacting the mouthpiece 1556 at the notch 1570 and with the mouthpiece 1556 contacting and depressing the tongue of the patient. In addition, when the oropharyngeal device 1550 is placed the handle 1554 is positioned outside of the patient's mouth so that the endotracheal tube 1552 can be secured to the oropharyngeal device 1550 (e.g., at the handle 1554 of the oropharyngeal device 1550). In some configurations, when the oropharyngeal device 1550 is placed, the distal end 1572 of the curved section 1566 is positioned within the throat (e.g., proximal to including above the larynx). Thus, a distal end of the conduit 1558 is positioned within the throat (e.g., proximal to including above the larynx). In this way, oxygen delivered from the port connector 1560 flows through the conduit 1558 and is emitted out into the throat of the patient to deliver oxygen to the airway of the patient (even during placement of the device).

In some embodiments, including after the oropharyngeal device 1550 has been placed, a securing device (e.g., the securing device 1500) can be placed into engagement with the patient. Then, the oropharyngeal device 1550 can be coupled to securing device. For example, the oropharyngeal device 1550 (e.g., the handle 1554 of the oropharyngeal device 1550) can be coupled to the bracket 1514 thereby securing the oropharyngeal device 1550 to the securing device. In some cases, the oropharyngeal device 1550 can include a clip that can couple (and decouple) the oropharyngeal device 1550 from the securing device (e.g., the clip coupling the handle 1554 of the oropharyngeal device 1550 to the bracket of the securing device).

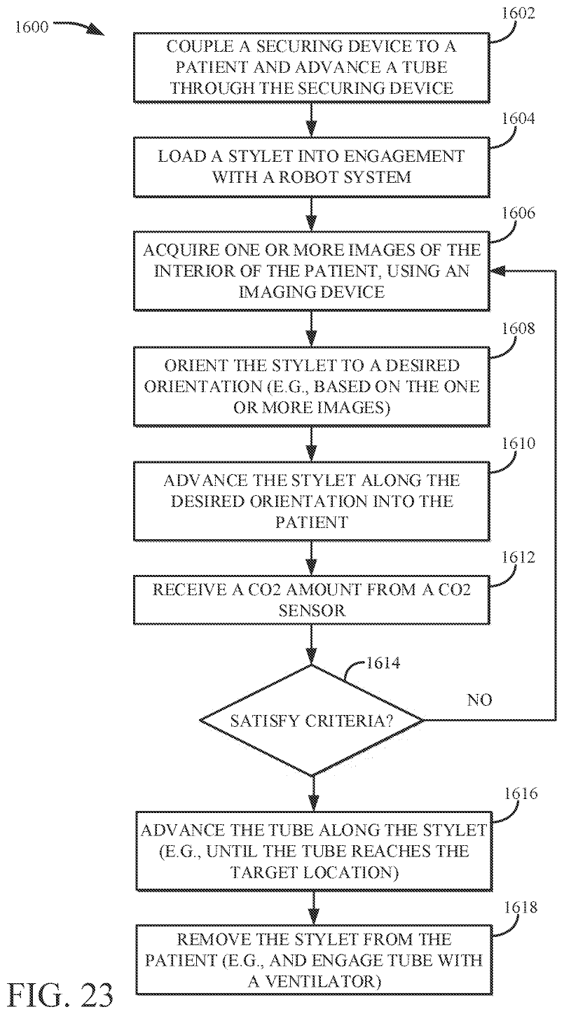

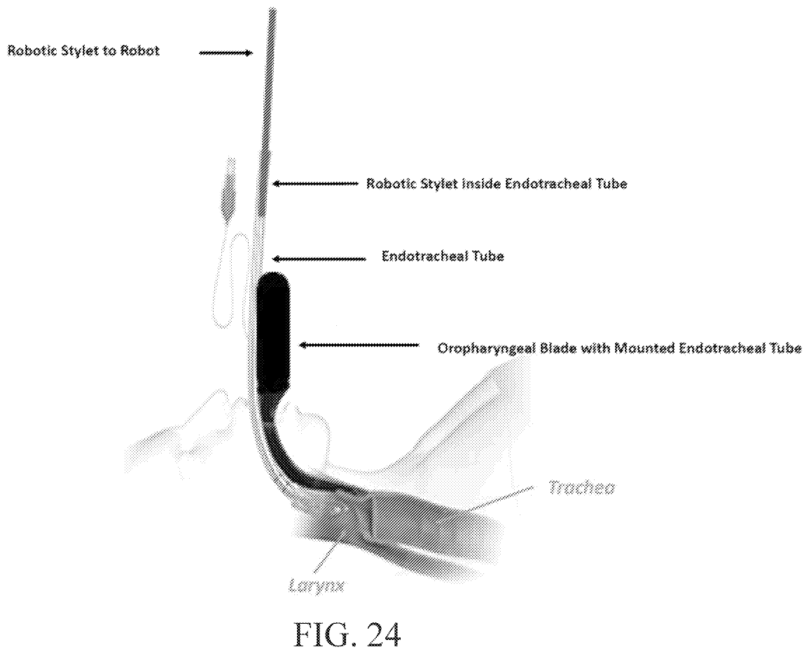

FIG. 23 shows a flowchart of a process 1600 for guiding a stylet (or in other words a guide wire) to a target location within a patient. In some cases, the process 1600 can include guiding a catheter (or other medical device) to the target location within the patient (e.g., after the guidewire, and in particular the distal end of the guidewire, is at the target location). In some cases, the process 1600 can be implemented using one or more computing devices, as appropriate, which can include a controller, other processing circuity (e.g., a GPU), etc. IN addition, the process 1600 can be implemented using any of the systems described herein as appropriate. In some cases, however, portions of the process 1600 can be implemented completely by the robot system, as appropriate, without aid from other computational resources. In this way, the process 1600 can be advantageously performed in locations without internet, cellular, or other communication access (e.g., on a battlefield, in a remote area, etc.). In some cases, some or all portions of the process 1600 can be completed when the patient is in an emergency vehicle (e.g., an ambulance), which expedite treatment for the patient when the patient arrives at the medical facility.

At 1602, the process 1600 can include placing an oropharyngeal device (e.g., the oropharyngeal device 1550) into the patient's mouth and throat as described above. For example, this can include inserting the distal end of the oropharyngeal device into the mouth of the patient, and into the throat of the patient so that the proximal end of the oropharyngeal device (e.g., including the handle of the oropharyngeal device) is positioned outside of the patient (e.g., outside of the mouth of the patient). In some cases, this can include contacting (and depressing) the tongue of the patient, and contacting the structures that define the larynx with a distal end of the oropharyngeal device thereby further opening the laryngeal cavity. In some embodiments, including when the oropharyngeal device has been placed, the block 1602 can include coupling an oxygen source (e.g., a pressurized oxygen source, including a pressurized tank of oxygen) to a port connector of the oropharyngeal device. Then, the block 1602 can include (a computing device) delivering oxygen into the port connector of the oropharyngeal device, through (and out) the conduit of the oropharyngeal device into the throat of the patient (e.g., proximal to the larynx). In some cases, and advantageously, with the oropharyngeal device placed, oxygen gas can be delivered into the patient's throat during the entire process 1600. Thus, oxygen gas can be delivered to the patient (e.g., in this manner) during each block of the process 1600.

The block 1602 can include coupling a securing device (e.g., an endotracheal tube securing device) to a patient. In some cases, this can include adhering one or more pads of the securing device to the patient (e.g., a first pad to the patient's first cheek, and a second pad to a patient's second cheek). In some embodiments, including when the oropharyngeal device has been placed, the block 1602 can include coupling the oropharyngeal device to the securing device (e.g., with a clip, a tie, etc.). In some embodiments, including when the oropharyngeal device and the securing device have been placed, the block 1602 can include advancing a tube (e.g., an endotracheal tube) into an orifice of the patient (e.g., the mouth and down the throat of the patient) until the distal end of the tube reaches the pharynx of the patient. In some configurations, this can include inserting the tube through a hole in the securing device, placing the tube into contact with a channel of the oropharyngeal device, and advancing the tube along the channel of the oropharyngeal device until the distal end of the tube reaches the pharynx of the patient. In some cases, including once the tube has been placed, the block 1602 of the process 1600 can include locking the tube to the securing device to block relative movement between the tube and the securing device. For example, this can include rotating (or advancing) a lock of the securing device until the lock contacts the tube. In some cases, this can include coupling the tube to the oropharyngeal device to block relative movement between the oropharyngeal device and the tube. For example, this can include tying (using one or more ties), clipping (using one or more clips), etc., the oropharyngeal device to the tube (e.g., to temporarily secure the tube to the oropharyngeal device).

At 1604, the process 1600 can include loading a stylet into engagement with a robot system. In some cases, this can include coiling the stylet into a coiled configuration around a protrusion, advancing a proximal end of the stylet into a hole of the robot system. In some cases, this can include closing a cartridge that includes the stylet (e.g., in a coiled configuration) into engagement with a main body of a housing of the robot system. In some cases, this can include optically coupling an optical bundle of the stylet with one or more optical components of the robot system. For example, this can include optically coupling a proximal end of a first optical fiber of the optical bundle to an imaging device of the robot system, and optically coupling a second optical fiber of the optical bundle to a spectrometer of the robot system. In some cases, this can include optically coupling a proximal end of a light guide to an illumination source of the robot system. In some configurations, this can include coupling a proximal end of each filament to an extender of a respective actuator of the robot system.

In some configurations, the block 1604 of the process 1600 can include advancing the stylet through the hole of the securing device and into the interior of the patient (e.g., the mouth of the patient and into the throat of the patient).

At 1606, the process 1600 can include a computing device acquiring one or more images of the interior of the patient, using the imaging device of the robot system. For example, light from the interior of the patient can be directed into the distal end of an optical fiber of the optical bundle (e.g., by being focused by a lens), can travel back through the optical fiber, and can be directed at the imaging device to generate imaging data for the one or more images. In some cases, while the computing device acquires imaging data, a computing device can cause an illumination source to illuminate the interior of the patient thereby illuminating the field of view fo the imaging device.

At 1608, the process 1600 can include a computing device orienting the stylet to a desired orientation (e.g., based on the one or more images acquired from the block 1606). In some cases, this can include a computing device analyzing the one or more images. For example, a computing device can input the one or more images into a machine learning model that has been trained to identify an anatomical region of interest (e.g., structures of the anatomical region of interest indicative of the respiratory tract). Then, a computing device can receive from the machine learning model, an indication whether or not the anatomical region of interest was identified within the one or more images, and if so, a size of the identified anatomical region of interest (e.g., the machine learning model segmenting out the identified anatomical region of interest from the one or more images), which can be indicative of the distance the distal end of the stylet is away from the anatomical region of interest. Thus, in some cases, a computing device can determine a distance from the distal end of the stylet and to the anatomical region of interest, based on the size of the identified anatomical region of interest within the one or more images. In some embodiments, a computing device can determine the desired orientation from the identification of the anatomical region of interest and the distance the anatomical region of interest is from the distal end of the stylet. For example, the location of the identified anatomical region relative to a center of the one or more images can indicate which direction to orient the stylet to center the anatomical region onto the center of the one or more images. In addition, the distance can correspond to the amount of the adjustment in orientation. For example, the smaller the identified anatomical region of interest, the farther the distal end of the stylet is from the anatomical region of interest and thus the smaller the magnitude of the desired orientation that is required for compensating and aligning the distal end of the stylet.

In some embodiments, the block 1608 of the process 1600 can include a computing device moving an extender of one or more actuators thereby adjusting the tensile loading on the one or more filaments of the stylet until the current orientation of the stylet aligns with the desired orientation.

At 1610, the process 1600 can include a computing device advancing the stylet along the desired orientation into the patient. For example, this can include a computing device causing a motor to rotate a protrusion that receives a coil of the stylet thereby forcing the stylet to advance further into the patient (e.g., further into the throat of the patient). At 1612, the process 1600 can include a computing device receiving a CO2 amount value from a CO2 sensor (e.g., of the stylet) that is positioned within the interior of the patient.

At 1614, the process 1600 can include a computing device determining whether or not one or more criteria have been satisfied. If at the block 1614, the computing device determines that the one or more criteria have not been satisfied then the process 1600 can proceed back to the block 1606 to acquire additional images using the imaging device. Alternatively, if at the block 1614, the computing device determines that the one or more criteria have been satisfied, then the process 1600 can proceed to the block 1616 to advance the tube along the stylet. In some embodiments, a first criteria can be the result of a comparison of the CO2 amount value to a threshold value. For example, the first criteria can be satisfied based on the CO2 amount value (e.g., at the block 1612) exceeding a threshold value. As a more specific example, relatively higher CO2 amount values indicate that the distal end of the stylet is positioned within the trachea (as compared with the esophagus). In this case, if the computing device determines that the CO2 amount value is greater than the threshold value, then the first criteria can be satisfied (e.g., if the target region requires traveling into the trachea). As another example, relatively lower CO2 amount values indicate that the distal end of the stylet is positioned within the esophagus (as compared with the trachea). In this case, if the computing device determines that the CO2 amount value is less than a threshold value, then the computing device can determine that the first criteria is satisfied (e.g., when the target region requires traveling into the esophagus).

In some embodiments, a second criteria can be the identification of the tracheal bifurcation. For example, a computing device can receive the one or more images (or can acquire an additional image after, for example, the one or more images have been acquired), which can be used to identify the tracheal bifurcation (or in other words the tracheal carina). This can be similar to the block 1608 of the process 1600 in which a computing device can input the one or more images (or the additional image) into a machine learning model trained to identify (and segment out) the tracheal bifurcation. In some cases, if the machine learning model identifies the tracheal bifurcation (e.g., by the presence of a segmented image from the one or more images) the computing device can determine a size of the tracheal bifurcation in the image (e.g., the segmented image in which the tracheal bifurcation has been identified). Then, a computing device can determine the distance between the tracheal bifurcation and the distal end of the stylet based on the size of the tracheal bifurcation in the image (e.g., because the smaller the size of the tracheal bifurcation in the image, the farther the distal end of the stylet is from the tracheal bifurcation).

In some configurations, a third criteria can be the result of the comparison of the distance between the tracheal bifurcation and the distal end of the stylet to a threshold value (e.g., 3 mm). For example, a computing device can demine that the third criteria is satisfied, based on this distance being less than or equal to the threshold value. In some embodiments, if the one or more criteria have been satisfied at the block 1614, then a computing device can notify a practitioner (e.g., by presenting an indication on a display) to indicate that the distal end of the device is a the target region.

At 1616, the process 1600 can include advancing the tube along the stylet (e.g., until the tube reaches the target location). In some cases, this can include decoupling the tube from the securing device to allow relative movement between the tube and the securing device. In some configurations, this can include decoupling the tube from the oropharyngeal device to allow relative movement between the tube and the oropharyngeal device. In some cases, this can include removing one or more clips from engaging between the tube and the oropharyngeal device, removing one or more ties from engagement between the tube and the oropharyngeal device, etc. Once the tube is free to move, the tube can be advanced along the stylet until a distal end of the tube reaches or is proximal to the distal end of the stylet. In some embodiments, a computing device can determine that a distal end of the tube has reached a location that overlaps or is proximal to the a distal end of the stylet. For example, as the tube is advanced closer to the distal end of the stylet less light from the illumination source (e.g., emitted out the distal end of the stylet using the light pipe) is directed into the distal end of the optical bundle. Thus, if a computing device is unable to acquire an image, via receiving light from an optical fiber of the optical bundle, or if one or more pixel values of an image (e.g., an average pixel values of the image) of the interior of the patient exceeds (e.g., is lower than) a threshold value, then the computing device can determine that the distal end of the tube is at the target location. In this case, then a computing device can notify a practitioner by, for example, presenting a graphic on a display, to indicate that the tube has been ideally placed.

In some embodiments, the block 1616 can include locking the tube to the securing device (again) to block relative movement between the tube and the securing device (e.g., to lock the tube so that the distal end of the tube is at the target location). In some cases, the block 1616 can include confirming placement of the tube. For example, this can include retracting the distal end of the stylet an amount (e.g., 1 cm) by for example, rotating the motor in the opposing rotational direction to re-coil the stylet, and acquiring an image in a similar manner as acquiring the one or more images at the block 1606. This image can be presented on a display to be verified by a practitioner. In some cases, once the distal end of the tube has been confirmed to be placed correctly (after receiving a user input indicating correct placement), the process can proceed to the block 1618.

At 1618, the process 1600 can include a computing device removing the stylet from the patient. For example, this can include retreating the stylet back through the patient. In some cases, the computing device can utilize previous actuator commands used to drive extension of the stylet into the patient. For example, the actuator commands can be reversed to drive retraction of the stylet. In other cases, the blocks 1606-1610 can be repeated until the stylet is retracted a desired amount, except that inserted of advancing the stylet (e.g., at the block 1610), the stylet is retracted. In still other cases, the block 1618 can include manually removing the stylet from the tube, and out through the securing device. In some embodiments, the block 1618 can include engaging a ventilator with the tube to begin ventilating the patient.

FIG. 24 shows a schematic illustration of an endotracheal tube inserted into the throat of the patient and with a stylet having been inserted into the endotracheal tube.

### EXAMPLE

The following is a non-limiting example in accordance with embodiments of the invention.

### Experimental Data Support

The process of the autonomous endotracheal tube insertion was validated by dividing it into two individual parts: an object detection functionality that guides the robot and an integrated system that controls the robot individually.

### Image Dataset (for Robot Guidance Experiment) and Phantom Model (for Robot Control Experiment)

A commercially available training model, Koken Model for Suction and Tube Feeding Simulator: LM-097B (Koken Co Ltd, Bunkyo-ku, Japan), was purchased for experimentation. The images used for training of the model in tracheal detection were obtained utilizing this phantom. These images were obtained by manual control and automatic control of the robot during image/data gathering.

### System Configuration

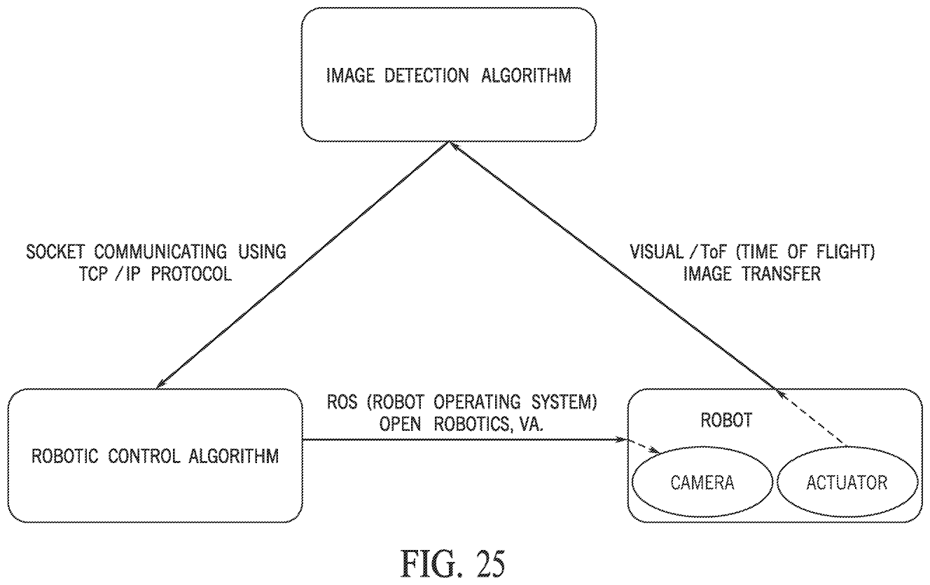

A macroscopically one-way closed loop system was built, consisting of 1) robot, 2) robot controlling computer, and 3) tracheal detection computer. The robot was controlled with communication based on ROS™ (Open Robotics, Mountain View, CA and Symbiosis, Singapore) from the robot controlling computer. The robot controlling computer transitioned between multiple modes based on the information provided by the trachea detection computer via primitive socket communication. The trachea detection computer received stream images from the camera which the robot carried (FIG. 25).

### Robot Guidance

An AI based detection and tracking model was developed which enables the robot to traverse autonomously using the real-time captured images. The objective is to first detect the trachea opening from the images and then follow the path predicted by a detection-tracking based mechanism. For detection, a deep learning-based detector (YOLO) was trained to detect the trachea, by further distinguishing between the esophageal and tracheal openings. For tracking, we specifically use a fast and computationally efficient median filtering technique.

### A. Trachea Detection

Convolutional neural network (CNN) based detectors have achieved a state-of-the art performance for real-time detection tasks. Different from traditional methods of pre-defined feature extraction coupled with a classifier, these convolutional neural network-based detection algorithms are designed by a unified hierarchical representation of the objects that are learned using the imaging data. These hierarchical feature representations are achieved by the chained convolutional layers which transform the input vector into a high dimensional feature space. For esophageal detection, we used a 26-layer CNN-based detection model. The first 24 layers are fully convolutional layers are pre-trained on an Imagenet dataset and the last two layers are fully connected layers which output the detected regions. Our variant of the 26-layer CNN-based detection model is fine-tuned with the colored images of the nasogastric regions.

### B. Tracking

A median flow filtering based tracking technique was designed to predict the motion vector for the robotic tube, where median filtering in a classical tracking technique.

### C. Robotic Control

The object detection via YOLOv3 and the object tracking via median flow tracker was implemented with Python 3.7.6. The environment was built on Ubuntu 18.04. As for Graphics Processing Unit (GPU) integration, cuDNN 7.6.5 and CUDA toolkit 10.0 were served for use.

The training part was implemented by feeding the annotated image to Keras implementation of YOLOv3. The version for Keras was 2.2.4 and this version runs TensorFlow 1.15 on the backend. The dataset was created with an annotation software, VoTT R (Microsoft, Redmond, WA).

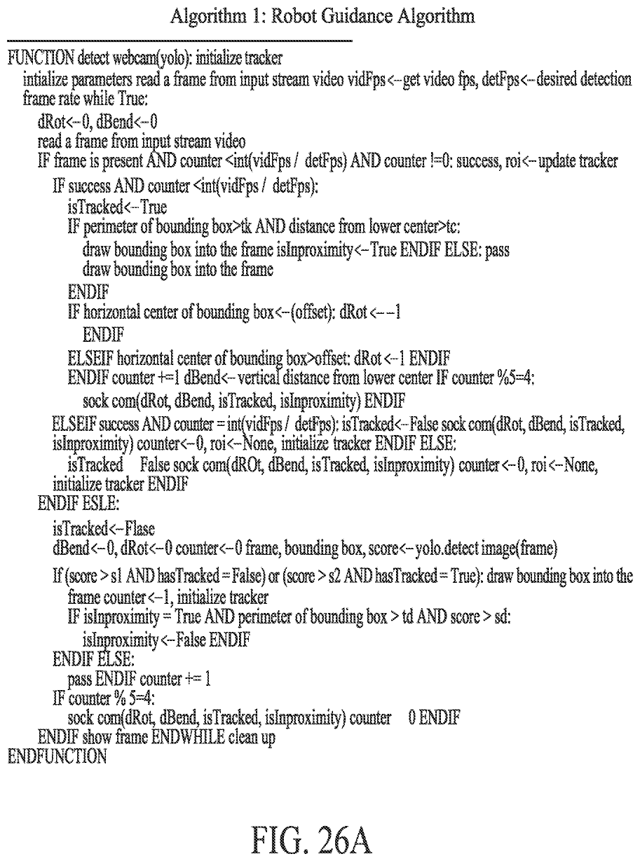

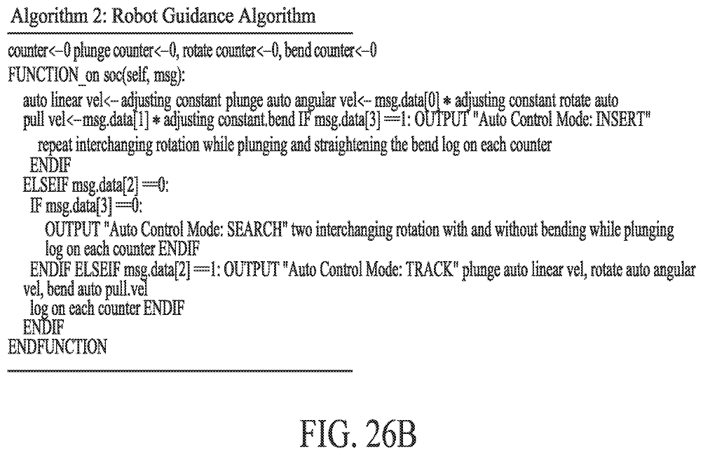

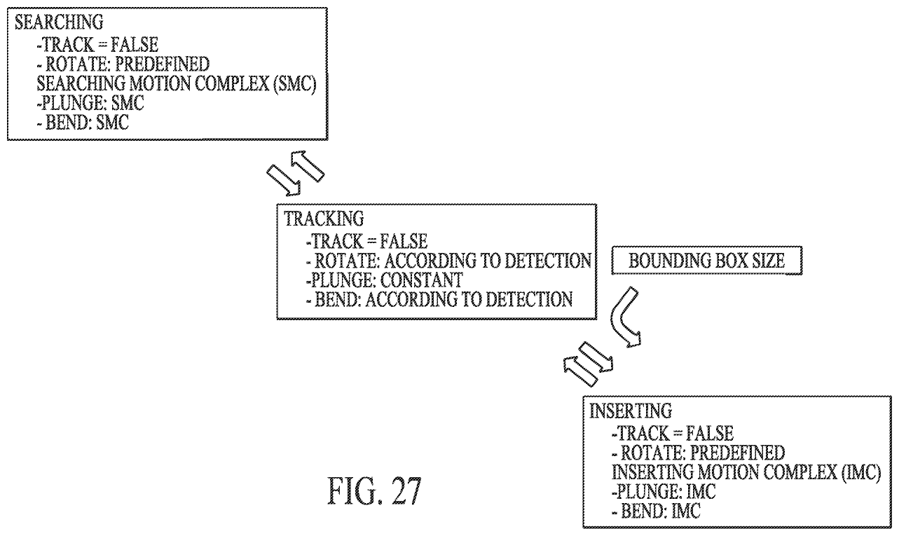

We adopted a learning rate for 1000 training epochs and saved the model parameters every 100 epochs. Among the saved models, the one that achieved the highest Average Precision (AP) for Intersection over Union (IoU) of 50% or higher was considered as positive on the validation set and was therefore selected as the final model to be evaluated on the testing set. The detection part was also implemented based on Keras 2.2.4 running TensorFlow 1.15 on the backend. As for the tracking part, tracking API in OpenCV 4.1.0 was used. The bounding box detected by YOLOv3 was passed to Median Flow tracker at a 1:5 ratio, thereby realizing real-time detection, tracking and control using two families of algorithms (FIGS. 26A, 26B). The distance of the robot to the trachea was approximated by the size of the detected bounding box. A transitioning strategy was built between certain modes each aimed at searching the trachea, tracking the trachea, and inserting the guide into the trachea, respectively (FIG. 27).

### Experimental Model

The system was evaluated by dividing the robotic endotracheal intubation process into two individual phases. One is guidance and detection and the other is control.

### Robot Guidance Validation

An evaluation was conducted to determine whether the CNN-based object detection of our system can detect real trachea. Endoscopic images with clearly open trachea and clearly closed trachea with more than two thirds of the aspect is visible were picked. The obtained images were incorporated into the YOLOv3 training described earlier in the Robot Guidance section.

Accuracy in recognizing the trachea compared to human recognition was evaluated using mean Average Precision (mAP) and Average Precision (AP). Additionally, Precision-Recall curve for each detection class was depicted.

### Robot Control Validation

Here, it was evaluated if the robot can control itself to the trachea. The training was conducted in an identical way as the Robot Guidance Validation experiment. It was evaluated if an endotracheal tube can actually travel over the robot through to the trachea by comparing the success rate of the tube reaching trachea with and without robot inside the trachea.

The success rate of the endotracheal tube in reaching the trachea was evaluated using a commercially-available endotracheal tube with an inner diameter of 7 mm.

### Results

A statistical analysis was utilized for the tube insertion part of the robot control validation experiment to evaluate the significance of the difference in the success rate between the proposed method and internal controls. Statistical analysis was conducted by Prism (GraphPad Software, San Diego, CA). Significance cutoff point was set to be 0.05. Power analysis was conducted to optimize the number of trials that were necessary for each experimental setup based on pilot experiments.

### Robot Guidance Validation Experiment

Accuracy of the detection with regard to mAP and AP were assessed for each datasets. The program algorithm demonstrated ability to detect the trachea in the closed configuration (97%) and in the opened configuration (100%).

sRobot Control Validation Experiment

The success rate of the robot to travel to the trachea was 96.7% (29/30) for fully integrated detection based control for fully the integrated detection based control, vs. 6.7% (2/30) for blind manual insertion, respectively.

Many modifications and variations to this preferred embodiment will be apparent to those skilled in the art, which will be within the spirit and scope of the invention. Therefore, the invention should not be limited to the described embodiment. To ascertain the full scope of the invention, the following claims should be referenced.

What is claimed:

1-21. (canceled)

22. A guidance system comprising:
an illumination source configured to illuminate an interior of the patient;
a robot system including an imaging device; and
a stylet configured to be inserted into an orifice of a patient, the stylet having a proximal end and a distal end, the stylet including an optical bundle having an optical fiber optically coupled to the imaging device; the optical fiber being configured to direct light from within the interior of the patient and to the imaging device.

23. The guidance system of claim 22, wherein the stylet includes a plurality of filaments coupled to or integrated within a body of the stylet,
wherein the robot system includes a plurality of actuators,
wherein each filament is coupled to an extender of a respective actuator, and
wherein extension and retraction of the extender of an actuator tensilely loads the respective filament to adjust the orientation of the stylet relative to the robot system.

24. The guidance system of claim 23, wherein the robot system includes a motor that is configured to rotate the stylet to advance a distal end of the stylet further into the patient.

25. The guidance system of claim 23, wherein the stylet includes a CO2 sensor,
wherein the robot system includes a controller in communication with the CO2 sensor, and
wherein the controller is configured to:
receive, using the CO2 sensor, a CO2 amount value; and
determine that a distal end of the stylet is at a target location within the patient, based on the CO2 amount value.

26. The guidance system of claim 25, wherein the controller is in communication with the illumination source and the imaging device, and
wherein the controller is further configured to:
cause the illumination source to emit light to illuminate the interior of the patient;
receive, using the imaging device, an image of the interior of the patient;
identify an anatomical region of interest within the image;
determine a desired orientation based on the identification of the anatomical region of interest within the image;
cause the plurality of actuators to adjust the stylet to be oriented at the desired orientation; and
advance the stylet further into the interior of the patient.

27. The guidance system of claim 26, wherein the controller is further configured to:
receive, using the imaging device, another image of the interior of the patient;
identify a tracheal bifurcation within the another image; and
determine that the distal end of the stylet is at the target location within the patient, based on the CO2 amount value exceeding a threshold value, and the identification of the tracheal bifurcation within the another image.

28. The guidance system of claim 23, wherein the stylet includes a channel,
wherein the robot system includes a gas source that is configured to be in fluid communication with the channel, and
wherein gas from the gas source is configured to be directed though and out the channel into the interior of the patient.

29. The guidance system of claim 23, wherein the stylet includes a channel,
wherein the robot system includes a vacuum source that is configured to be in fluid communication with the channel, and
wherein the vacuum source draws fluid out from the interior of the patient and through and out the channel.

30. The guidance system of claim 22, wherein the stylet includes:
a light pipe optically coupled to the illumination source, the light pipe directing light emitted from the illumination source into the interior of the patient; and
a lens optically coupled to a distal end of the optical fiber, the lens being configured to focus light from within the patient into the distal end of the optical fiber;

31. The guidance system of claim 22, wherein the stylet includes:
a channel;
a light pipe optically coupled to the illumination source, the illumination source being part of the robot system; and
a CO2 sensor, and
wherein the optical bundle, the light pipe, and the CO2 sensor each is positioned within the channel.

32. The guidance system of claim 23, further comprising an oropharyngeal device that is configured to be inserted into the mouth of the patient.

33. The guidance system of claim 32, wherein the oropharyngeal device includes a handle and a mouthpiece coupled to the handle, the handle having a cross-sectional height that is greater than a cross-sectional height of the mouthpiece, the mouthpiece having a curved section that curves away from a longitudinal axis of the oropharyngeal device,
wherein the mouthpiece is configured to be positioned inside the mouth of the patient when the oropharyngeal device is placed into the orifice of the patient, and
wherein the handle is configured to be positioned outside of the mouth of the patient when the oropharyngeal device is placed into the orifice of the patient.

34. The guidance system of claim 33, wherein the mouthpiece of the oropharyngeal device is configured to contact a tongue of the patient, and
wherein a distal end of the mouthpiece is configured to be positioned within the throat of the patient.

35. The guidance system of claim 33, wherein the oropharyngeal device includes:
a conduit extending through the handle and through the mouthpiece;
a port connector configured to interface with an oxygen gas source, the port connector being in fluid communication with the conduit, and
wherein oxygen gas from the oxygen gas source is configured to flow into the port connector, through and out the conduit into the throat of the patient.

36. The guidance system of claim 33, further comprising an endotracheal tube, a distal end of the endotracheal tube being configured to be inserted into the mouth and throat of the patient, and
wherein the endotracheal tube is configured to be removably coupled to the oropharyngeal device and a securing device that is configured to be coupled to the head of the patient.

37. A method of intubating a patient, the method comprising:
inserting a distal end of an oropharyngeal device into the mouth of the patient and into the throat of the patient;
advancing a distal end of an endotracheal tube along the oropharyngeal device until the distal end is positioned within the throat of the patient;
coupling the endotracheal tube to the oropharyngeal device; and
inserting a distal end of a stylet into the endotracheal tube until the distal end of the stylet reaches a target location inside the trachea of the patient;

38. The method of claim 37, further comprising:
decoupling the endotracheal tube from the oropharyngeal device;
advancing the distal end of the endotracheal tube along the stylet until the distal end of the endotracheal tube overlaps with or is proximal to the distal end of the stylet;
retracting the stylet back through the endotracheal tube until the entire stylet is outside of the patient; and
engaging a ventilator with the proximal end of the endotracheal tube.

39. The method of claim 37, further comprising introducing oxygen gas, from a pressurized oxygen gas source, through a port connector of the oropharyngeal device, through a conduit of the oropharyngeal device, and into the throat of the patient during the insertion of the stylet into the endotracheal tube until the distal end of the stylet reaches the target location.
